(19) United States
(12) Patent Application Publication
(10) Pub. No.: US 2016/0068818 A1
(43) Pub. Date: Mar. 10, 2016

(54) A METHOD FOR GENERATING INDUCED PLURIPOTENT STEM CELLS

(71) Applicant: Glykos Finland Oy, Helsinki (FI)

(72) Inventors: Milla MIKKOLA, Helsinki (FI); Timo OTONKOSKI, Helsinki (FI); Heli Mononen, Helsinki (FI); Tero SATOMAA, Helsinki (FI); Juhani SAARINEN, Helsinki (FI)

(73) Assignee: Glykos Finland Oy, Helsinki (FI)

(21) Appl. No.: 14/785,020

(22) PCT Filed: Apr. 16, 2014
(86) PCT No.: PCT/FI2014/050276

§ 371 Date: Oct. 16, 2015

(30) Foreign Application Priority Data
Apr. 16, 2013 (FI) 20135370
Jun. 11, 2013 (FI) 20135637

(51) Int. Cl. C12N 5/074; G01N 33/569

(52) U.S. Cl. CPC C12N 5/0696; G01N 33/56966; C12N 2506/1307

(57) ABSTRACT

The invention relates to a method for generating induced pluripotent stem cells, wherein the method comprises: step a) of inducing non-pluripotent cells to produce a mixture comprising induced pluripotent stem cells and non-pluripotent cells; step b) of contacting the mixture comprising induced pluripotent stem cells and non-pluripotent cells obtainable from step a) with a binding agent, wherein the binding agent is capable of binding an epitope consisting of the non-reducing terminal saccharide structure according to formula (Fucα1-2)ₐGalβ1-3HexNAcβ, wherein a=0 or 1 and Hex is either Glc or Gal; and step c) of selecting an induced pluripotent stem cell bound by the binding agent.

### FIELD OF THE INVENTION

The invention relates to a method for generating induced pluripotent stem cells. The invention also relates to an induced pluripotent stem cell or an induced pluripotent stem cell population obtainable by the method, to a composition, to a kit and to a culture system.

### BACKGROUND OF THE INVENTION

Induced pluripotent stem cells (iPS cells) have great potential in various lines of developmental and genetic research and regenerative medicine.

However, current methods of generating induced pluripotent stem cells from non-pluripotent cells such as human dermal fibroblasts involve complex, time-consuming and expensive procedures. Stable iPS cell lines are produced at low efficiency: typically less than 1% of induced non-pluripotent cells become fully reprogrammed induced pluripotent stem cells. Current methods of generating induced pluripotent stem cells often utilise manual isolation of candidate colonies after induction, which is labour-intensive, time-consuming and inefficient. Individual colonies are typically selected and passaged to several rounds of cultivation before expansion.

Induced pluripotent stem cells are also highly sensitive to culture conditions, and there are several technical issues involved in their cultivation. One of the major problems of the traditional methods and culture systems is that the use of stem cells or extracts in clinical applications is hampered by the presence of material derived from animals present in complex matrices in the culture. The current culture methods are also laborious and difficult to scale, and there is always a need to culture cells more efficiently, more cost-effectively and in larger numbers or quantities. It is frequently difficult to maintain the stem cells as undifferentiated and in uniform quality.

Furthermore, even after extensive selection, induced pluripotent stem cells obtained using conventional methods may be heterogeneous. Improved methods for fast, efficient and/or specific selection and cultivation of fully reprogrammed induced pluripotent stem cells are thus needed.

### PURPOSE OF THE INVENTION

The purpose of the present invention is to provide methods for generating induced pluripotent stem cells.

### SUMMARY

The method for generating induced pluripotent stem cells according to the present invention is characterized by what is presented in claim 1.

The induced pluripotent stem cell or induced pluripotent stem cell population according to the present invention is characterized by what is presented in claim 27.

The composition according to the present invention is characterized by what is presented in claim 29.

The composition according to the present invention for use as a medicament is characterized by what is presented in claim 30.

The kit according to the present invention is characterized by what is presented in claim 31.

The culture system according to the present invention is characterized by what is presented in claim 39.

### BRIEF DESCRIPTION OF THE DRAWINGS

The accompanying drawings, which are included to provide a further understanding of the invention and constitute a part of this specification, illustrate embodiments of the invention and together with the description help to explain the principles of the invention. In the drawings:

FIG. 1 shows fluorescence-activated cell sorting results and subsequent forming of induced pluripotent stem cell colonies. Human fibroblasts were retrovirally infected and sorted with K21 and Tra-1-60 antibodies at day 15 (A) and day 10 (B) after infection. K21 positive cells at day 15 after infection were able to form iPS colonies in culture (C);

FIG. 2 demonstrates analyses of basic characteristics of stem cells cultivated;

FIG. 3 shows results of clonogenicity and cell growth assays of stem cells;

FIG. 4 demonstrates validation of binding specificity of stem cells;

FIG. 5 demonstrates human pluripotent stem cells growing as a cell layer and without colony formation on culture surface coated with ECA lectin;

FIG. 6 shows a comparison of reprogrammed and FACS sorted Tra-1-60+/K21+ double positive cells and Tra-1-60+ single positive cells after sorting; and

FIG. 7 shows results of analysis of iPS cell lines derived from retrovirally reprogrammed fibroblasts either by FACS sorting of Tra-1-60+/K21+ double positive cells or by manual picking of emerged colonies.

### DETAILED DESCRIPTION OF THE INVENTION

The present inventors have surprisingly found that one or more embodiments of the present invention allow for high efficiency of generation of induced pluripotent stem cells.

In this context, the term “induced pluripotent stem cell” should be understood as referring to a pluripotent stem cell derived from any non-pluripotent or differentiated cell, such as an adult somatic cell (e.g. a fibroblast or a blood cell), that has been induced so as to have all essential features of embryonic stem cells.

In one embodiment, the term “induced pluripotent stem cell” should be understood as referring to a human induced pluripotent stem cell.

The present invention relates to a method for generating induced pluripotent stem cells, wherein the method comprises:

step a) of inducing non-pluripotent cells to produce a mixture comprising induced pluripotent stem cells and non-pluripotent cells;

step b) of contacting the mixture comprising induced pluripotent stem cells and non-pluripotent cells obtainable from step a) with a binding agent,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal; and

step c) of selecting an induced pluripotent stem cell bound by the binding agent.

The inducing of non-pluripotent cells in step a) may be performed in various ways known in the art. For instance, non-pluripotent cells may be induced by forced expression of stem cell associated genes, such as the transcription factors (reprogramming factors) Oct-3/4, Sox2, Klf4, NANOG, Lin28, and c-Myc. The forced expression of the stem cell associated genes may be achieved e.g. by transfecting the non-pluripotent cells with a suitable vector. Suitable vectors may be e.g. retroviral, lentiviral, adenoviral, Sendai virus, transposon, plasmid vectors or naked DNA. Non-pluripotent cells may be induced by transient transfection of synthetic mRNAs encoding Oct-3/4, Sox2, Klf4, c-myc, L-myc, NANOG, and/or Lin28. Non-pluripotent cells may be induced by culturing the non-pluripotent cells with recombinant transcription factor proteins.

In one embodiment, non-pluripotent cells are induced in step a) using extra-chromosomal programming elements.

Episomal vectors, Sendai virus comprising of or mRNAs encoding one or more reprogramming factors, or small molecules may be considered to be “extra-chromosomal programming elements”. Extra-chromosomal programming elements may include RNA molecules (such as mRNA), DNA molecules (such as extra-chromosomally replicating vectors or vectors capable of replicating episomally), proteins, or small molecules. Treatment with small molecules, such as ALK5 inhibitor SB431412, MEK inhibitor PD0325901, valproic acid or thiazovivin, may also be performed in step a).

The extra-chromosomal programming elements may exit the induced pluripotent stem cells or they may be removed from the induced pluripotent stem cells after induction. Thus the induced pluripotent stem cell obtainable from step c) and/or step d) may no longer contain the extra-chromosomal programming element.

In one embodiment, non-pluripotent cells are induced in step a) using extra-chromosomal programming elements; and the extra-chromosomal programming elements are not present in the induced pluripotent stem cell obtainable from step c) and/or step d).

Non-pluripotent cells may also be induced using the method described in Miyaoka et al., Nature Methods 2014, 11 (3), 291-293.

In one embodiment, non-pluripotent cells are induced by transfecting the non-pluripotent cells with Oct-4, Sox2, Klf4 and c-Myc.

In one embodiment, non-pluripotent cells are induced by transfecting the non-pluripotent cells with Oct-4, Sox2, NANOG and LIN28.

In one embodiment, non-pluripotent cells are induced by transfecting the non-pluripotent cells with Oct-4 and Sox2 and by treating with valproic acid.

In one embodiment, non-pluripotent cells are induced by transfecting the non-pluripotent cells with Oct-4, Sox2, Klf4 and miRNA.

In one embodiment, non-pluripotent cells are induced by transient transfection of synthetic mRNAs encoding Oct-3/4, Sox2, Klf4, c-myc, L-myc, NANOG, and/or Lin28.

In one embodiment, non-pluripotent cells are induced by transfecting the non-pluripotent cells with Oct-4, Sox2, Klf4 and c-Myc and treating with MEK inhibitor PD0325901 or thiazovivin.

In one embodiment, non-pluripotent cells are induced by transfecting the non-pluripotent cells with Oct-4 and Klf4 and treating with MEK inhibitor PD0325901 and histone methyl transferase inhibitor BIX01294.

In one embodiment, non-pluripotent cells are induced by transfecting the non-pluripotent cells with Oct-4, Sox2, Klf4 and c-Myc and treating with sodium butyrate, SB431542, and PD0325901.

In one embodiment, non-pluripotent cells are induced by transducing the non-pluripotent cells with the Oct4, Sox2, Klf4 and c-Myc/L-myc proteins.

The non-pluripotent cells may be any non-pluripotent cells that may be induced to induced pluripotent stem cells.

In one embodiment, the non-pluripotent cells are human somatic cells.

In one embodiment, the non-pluripotent cells are human adult somatic cells.

In one embodiment, the non-pluripotent cells are human adult fibroblasts.

In one embodiment, the non-pluripotent cells are human white blood cells.

In one embodiment, the non-pluripotent cells are human CD34+ cells.

In one embodiment, the non-pluripotent cells are human peripheral blood mononuclear cells.

In one embodiment, the non-pluripotent cells are fibroblasts, keratinocytes, stem cells, hematopoietic cells, mesenchymal stem cells, mesenchymal cells, adipose cells, endothelial cells, neural cells, muscle cells, mammary cells, liver cells, kidney cells, skin cells, digestive tract cells, cumulus cells, gland cells, CD34+ cells, peripheral blood mononuclear cells, or pancreatic islet cells.

In one embodiment, the binding agent according to one or more embodiments described in this document is not capable of binding the non-pluripotent cells. In this embodiment, the epitope which the binding agent is capable of binding is not present in or on the surface of the non-pluripotent cells. It is currently known that binding agents capable of binding the epitope defined herein, such as K21, do not bind any, or an overwhelming majority of, non-pluripotent cells. The method according to the invention therefore is capable of generating induced pluripotent stem cells with very high efficiency of most, and potentially all, non-pluripotent cells amenable for induction.

In one embodiment, the method is directed to generating human induced pluripotent stem cells.

The efficiency of the induction in step a) is typically low; thus it is to be understood that a mixture comprising induced pluripotent stem cells and non-pluripotent cells will be obtained by inducing non-pluripotent cells. Furthermore, the non-pluripotent cells in said mixture may also comprise induced cells that are only partially reprogrammed.

In one embodiment, step a) comprises inducing non-pluripotent cells to produce a mixture comprising induced pluripotent stem cells and non-pluripotent cells and cultivating the mixture comprising induced pluripotent stem cells and non-pluripotent cells obtained. The mixture comprising induced pluripotent stem cells and non-pluripotent cells may be cultivated for a time period. A suitable time period may depend greatly on the method of inducing non-pluripotent cells and e.g. on the type of non-pluripotent cells.

In one embodiment, the time period is at least 1 day, or at least 2 days, or at least 3 days, or at least 4 days, or at least 5 days, or at least 6 days, or at least 7 days, or at least 8 days, or at least 9 days.

In one embodiment, the time period is at least 10 days.

In one embodiment, the time period is at least 11 days.

In one embodiment, the time period is 5 to 30 days, or 10 to 30 days, or 11 to 30 days, or 11 to 15 days, or 11 to 13 days.

In one embodiment, the time period is 1 to 30 days, or 2 to 30 days, or 3 to 30 days, or 1 to 15 days, or 2 to 14 days, or 3 to 13 days, or 4 to 12 days, or 5 to 11 days, or 6 to 10 days, or 1 to 10 days, or 13 to 15 days.

In one embodiment, the time period is sufficient to allow for reprogramming of non-pluripotent cells to induced pluripotent stem cells.

In one embodiment, the time period is shorter than a time period required for the mixture comprising induced pluripotent stem cells and non-pluripotent cells to form colonies.

The present inventors have surprisingly found that a binding agent capable of binding an epitope consisting of the non-reducing terminal saccharide structure according to formula (Fucα1-2)ₐGalβ1-3HexNAcβ, wherein a=0 or 1 and Hex is either Glc or Gal, may selectively bind a subset of cells from the mixture comprising induced pluripotent stem cells and non-pluripotent cells obtainable from step a), and thus allow efficient selection of induced pluripotent stem cells. Said subset comprises a high proportion of induced pluripotent stem cells.

The present inventors have found that an epitope comprising the non-reducing terminal saccharide structure according to formula

(Fucα1-2)ₐGalβ1-3HexNAcβ,

wherein a=0 or 1 and Hex is either Glc or Gal is present in or on the surface of induced pluripotent stem cells and may be used to select induced pluripotent stem cells with high efficiency. The non-reducing terminal saccharide structures according to said formula are structurally related and derived from the same oligosaccharide biosynthetic pathway.

In one embodiment, the binding agent is capable of binding an epitope consisting of the non-reducing terminal disaccharide structure according to formula

Galβ1-3HexNAcβ,

wherein Hex is either Glc or Gal.

In one embodiment, the binding agent is capable of binding an epitope consisting of the non-reducing terminal trisaccharide structure according to formula

Fucα1-2Galβ1-3HexNAcβ,

wherein Hex is either Glc or Gal. This embodiment allows for selecting a subset of cells from the mixture comprising induced pluripotent stem cells and non-pluripotent cells obtainable from step a), and thus allows efficient selection of induced pluripotent stem cells. Said subset comprises a high proportion of induced pluripotent stem cells.

In this context, the abbreviation “Gal” should be understood as referring to D-galactose; “Hex” should be understood as referring to a hexose sugar; “HexNAc” should be understood as referring to N-acetylhexosamine; “Glc” should be understood as referring to D-glucose; “GlcNAc” should be understood as referring to N-acetyl-D-glucosamine (2-acetamido-2-deoxy-D-glucose); “GalNAc” should be understood as referring to N-acetyl-D-galactosamine (2-acetamido-2-deoxy-D-galactose); “Fuc” should be understood as referring to L-fucose; and all monosaccharides are in pyranose form.

The term “Lewis c blood group antigen” should be understood as referring to the disaccharide structure Galβ1-3GlcNAcβ.

The term “Tra+ cell” should be understood as referring to a cell which expresses the marker Tra-1-60. A Tra+ cell may be bound by the antibody Tra-1-60. The notation of said non-reducing terminal saccharide structure and the glycosidic bonds between the sugar residues comprised therein follows that commonly used in the art, e.g. “Galβ1-3HexNAcβ” should be understood as meaning a Gal residue linked by a covalent linkage between the first carbon atom of the Gal residue to the third carbon atom of the N-acetylhexosamine residue linked by an oxygen atom in the beta configuration, and that both monosaccharide residues are in β-anomeric pyranose form.

Glycolipid and carbohydrate nomenclature herein is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29).

In this context, the term “capable of binding” should be understood as referring to the ability of the binding agent to bind to an epitope consisting of the non-reducing terminal saccharide (di- or trisaccharide) structure. Binding to an epitope may be assessed by methods known in the art and/or methods described in the examples. As further examples only, binding, significant binding and/or kinetic measurements may be assayed by utilizing surface plasmon resonance-based methods on a Biacore apparatus, by immunological methods such as ELISA or by carbohydrate microarrays.

It should be understood that the binding agent may be capable of binding a molecule comprising other structures in addition to an epitope consisting of said non-reducing terminal saccharide (di- or trisaccharide) structure. For instance, said non-reducing terminal saccharide (di- or trisaccharide) structure may be bound to other saccharide residues or structures, to a protein moiety (a glycoprotein), to a lipid moiety (a glycolipid), or to any other structures present e.g. on the surface of an induced pluripotent stem cell. However, it is sufficient that the binding agent is capable of binding an epitope consisting of said non-reducing terminal saccharide (di- or trisaccharide) structure only, thus allowing highly specific selection of induced pluripotent stem cells.

In one embodiment, Hex is Glc. In other words, the binding agent is capable of binding an epitope consisting of the non-reducing terminal saccharide (di- or trisaccharide) structure according to the formula (Fucα1-2)ₐGalβ1-3GlcNAcβ, wherein a=0 or 1. This embodiment has the added utility that a binding agent capable of binding the non-reducing terminal saccharide structure according to the formula (Fucα1-2)ₐGalβ1-3GlcNAc is capable of binding highly specifically to induced pluripotent stem cells.

In one embodiment, Hex is Gal. In other words, the binding agent is capable of binding an epitope consisting of the non-reducing terminal saccharide (di- or trisaccharide) structure according to the formula (Fucα1-2)ₐGalβ1-3GalNAcβ, wherein a=0 or 1.

In one embodiment, the binding agent is capable of binding an epitope consisting of the non-reducing terminal trisaccharide structure according to formula Fucα1-2Galβ1-3GlcNAcβ.

In one embodiment, the binding agent is capable of binding an epitope consisting of the non-reducing terminal trisaccharide structure according to formula Fucα1-2Galβ1-3GalNAcβ.

In one embodiment, the binding agent is an antibody, a lectin, a carbohydrate-modifying protein, or a modification or a fragment thereof.

In one embodiment, the binding agent is an antibody or a lectin.

In one embodiment, the binding agent is an antibody or any modification or a fragment thereof. As an example only, the binding agent may be an scFv, a single domain antibody, an Fv, a VHH antibody, a diabody, a tandem diabody, a Fab, a Fab′, or a F(ab)2, provided it specifically binds said terminal non-reducing saccharide (di- or trisaccharide) structure. Furthermore, the antibody or a fragment thereof may be present in monovalent monospecific, multivalent monospecific, bivalent monospecific, or multivalent multispecific forms.

In one embodiment, the binding agent is an antibody.

In one embodiment, the binding agent is a monoclonal antibody.

The generation of an antibody capable of binding an epitope consisting of the terminal non-reducing saccharide (di- or trisaccharide) structure may be done using standard techniques well known in the art. For instance, in order to select antibodies capable of binding an epitope consisting of the terminal non-reducing saccharide structure, phage display based on standard procedures may be used as disclosed in e.g. “Phage Display: A Laboratory Manual”; Ed. Barbas, Burton, Scott & Silverman; Cold Spring Harbor Laboratory Press, 2001. Standard procedures of recombinant antibody technologies may be used to produce the antibody.

In one embodiment, the antibody is K21, anti-PLN, Hesca-2, A68-E/E3, A68-E/A2 or A68-B/A11.

In one embodiment, the antibody is a xeno-free K21, a xeno-free anti-PLN, a xeno-free Hesca-2, a xeno-free A68-E/E3, a xeno-free A68-E/A2 or a xeno-free A68-B/A11.

In one embodiment, the antibody is an antibody selected from the group consisting of K21, anti-PLN, Hesca-2, A68-E/E3, A68-E/A2, A68-B/A11, and any combination thereof.

These antibodies have the added utility that they are capable of binding an epitope consisting of said terminal non-reducing saccharide (di- or trisaccharide) structure with high specificity and/or selectivity.

In this context, the term “K21” should be understood as referring to the mouse IgM monoclonal antibody raised against human teratocarcinoma cell line Tera-1 (W. J. Rettig et al. 1985, Cancer Res. 45:815-821) and available from e.g. GeneTex (Irvine, Calif., USA; Cat. No. GTX23352), Abcam (Cambridge, UK; Cat. No. ab3352) and hybridoma cell line HB-8549 from the ATCC (Manassas, Va., USA). K21 is capable of binding the disaccharide Galβ1-3GlcNAcβ as well as glycoproteins and glycosphingolipids comprising the non-reducing terminal disaccharide Galβ1-3GlcNAcβ as the non-reducing end structure.

The term “anti-PLN” or “anti-PLN antibody” should be understood as referring to an antibody or a fragment thereof having the same binding specificity as K21.

In one embodiment, anti-PLN comprises the heavy chain complementarity determining regions HCDR1 of SEQ ID NO: 1, HCDR2 of SEQ ID NO: 2 and HCDR3 of SEQ ID NO: 3; and

the light chain complementarity determining regions LCDR1 of SEQ ID NO: 4, LCDR2 of SEQ ID NO: 5 and LCDR3 of SEQ ID NO: 6, or the light chain complementarity determining regions LCDR1 of SEQ ID NO: 7, LCDR2 of SEQ ID NO: 8 and LCDR3 of SEQ ID NO: 9.

In one embodiment, anti-PLN comprises the heavy chain complementarity determining regions HCDR1 of SEQ ID NO: 1, HCDR2 of SEQ ID NO: 2 and HCDR3 of SEQ ID NO: 3; and the light chain complementarity determining regions LCDR1 of SEQ ID NO: 4, LCDR2 of SEQ ID NO: 5 and LCDR3 of SEQ ID NO: 6.

In one embodiment, anti-PLN comprises the heavy chain complementarity determining regions HCDR1 of SEQ ID NO: 1, HCDR2 of SEQ ID NO: 2 and HCDR3 of SEQ ID NO: 3; and the light chain complementarity determining regions LCDR1 of SEQ ID NO: 7, LCDR2 of SEQ ID NO: 8 and LCDR3 of SEQ ID NO: 9.

In one embodiment, anti-PLN comprises a heavy chain variable region having a sequence that is at least 85%, or at least 90%, or at least 95%, or 100% identical to SEQ ID NO: 10.

In one embodiment, anti-PLN comprises a light chain variable region having a sequence that is at least 85%, or at least 90%, or at least 95%, or 100% identical to SEQ ID NO: 11.

In one embodiment, anti-PLN comprises a light chain variable region having a sequence that is at least 85%, or at least 90%, or at least 95%, or 100% identical to SEQ ID NO: 12.

In one embodiment, anti-PLN comprises a heavy chain variable region having a sequence that is at least 85%, or at least 90%, or at least 95%, or 100% identical to SEQ ID NO: 10, and a light chain variable region having a sequence that is at least 85%, or at least 90%, or at least 95%, or 100% identical to SEQ ID NO: 11 or to SEQ ID NO: 12.

In one embodiment, anti-PLN comprises a sequence that is at least 85%, or at least 90%, or at least 95%, or 100% identical to SEQ ID NO: 13.

In one embodiment, anti-PLN comprises a sequence that is at least 85%, or at least 90%, or at least 95%, or 100% identical to SEQ ID NO: 14.

In one embodiment, anti-PLN comprises a sequence that is at least 85%, or at least 90%, or at least 95%, or 100% identical to a sequence selected from the group consisting of SEQ ID NOs: 15-20.

In one embodiment, anti-PLN comprises a sequence that is at least 85%, or at least 90%, or at least 95%, or 100% identical to a sequence selected from the group consisting of SEQ ID NOs: 15-20; and a sequence that is at least 85%, or at least 90%, or at least 95%, or 100% identical to SEQ ID NO: 13 or 14.

Anti-PLN may also comprise a combination of any of the above embodiments.

The antibody or anti-PLN may be any type of antibody, e.g. (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a single domain antibody, (vi) a diabody or a tandem diabody, or (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv).

Furthermore, the antibody or a fragment thereof may be present in monovalent monospecific, multivalent monospecific, bivalent monospecific, or multivalent multispecific forms.

In one embodiment of the present invention, the antibody or anti-PLN is a human antibody. In this context, the term “human antibody”, as it is commonly used in the art, is to be understood as meaning antibodies having variable regions in which both the framework and complementary determining regions (CDRs) are derived from sequences of human origin.

In one embodiment, the antibody or anti-PLN is a humanized antibody. In this context, the term “humanized antibody”, as it is commonly used in the art, is to be understood as meaning antibodies wherein residues from a CDR of an antibody of human origin are replaced by residues from a CDR of a nonhuman species (such as mouse, rat or rabbit) having the desired specificity, affinity and capacity.

The generation of an antibody specifically binding the epitope according to the present invention may be done using standard techniques well known in the art. For instance, to select antibodies specifically binding said epitope, phage display based on standard procedures may be used as disclosed in e.g. “Phage Display: A Laboratory Manual”; Ed. Barbas, Burton, Scott & Silverman; Cold Spring Harbor Laboratory Press, 2001. Standard procedures of recombinant antibody technologies may be used to produce the antibody.

In one embodiment, the antibody or anti-PLN is a scFv, Fab, 20 F(ab′)2, or a single domain antibody.

In one embodiment, the antibody or anti-PLN is an IgG or IgM antibody.

In one embodiment, the antibody or anti-PLN is a recombinant antibody.

In one embodiment, the antibody or anti-PLN is a xeno-free antibody. In this context, the term “Hesca-2” should be understood as referring to the mouse IgM monoclonal antibody raised against human embryonic stem cell line BG-01v (M. G. Shoreibah et al. 2011, Stem Cells Dev. 20:515-25) and available from EMD Millipore (clone 060818-7A6, Cat. # MAB4406). Hesca-2 is capable of binding the disaccharide Galβ1-3GlcNAcβ as well as other related non-reducing end structures. In one embodiment, Hesca-2 is a xeno-free antibody.

In this context, the terms “A68-E/E3” (Code: 305) and “A68-E/A2” (Code: 306) should be understood as referring to the mouse IgG1 antibodies and the term “A68-B/A11” (Code: 304) to the mouse IgM antibody against Galβ1-3GalNAcβ disaccharide available from Glycotope (Berlin, Germany). In one embodiment, A68-E/E3, A68-E/A2, and/or A68-B/A11 is a xeno-free antibody.

In one embodiment, the antibody is selected from the group consisting of K21, anti-PLN, Hesca-2 and the combination thereof.

In one embodiment, the antibody is K21.

In one embodiment, the antibody is selected from the group consisting of A68-E/E3, A68-E/A2, A68-B/A11 and any combination thereof.

In one embodiment, the antibody is 17-206, anti-SSEA-5, A70-A/A9, A51-B/A6 or MBr1.

In one embodiment, the antibody is an antibody selected from the group consisting of 17-206, anti-SSEA-5, A70-A/A9, A51-B/A6, MBr1 and any combination thereof.

In this context, the term “17-206” should be understood as referring to the mouse IgG3 antibody against Fucα1-2Galβ1-3GlcNAcβ trisaccharide available from Abcam (Cambridge, UK).

In this context, the term “anti-SSEA-5” should be understood as referring to the mouse IgG1 antibody clone 8e11 against Fucα1-2Galβ1-3GlcNAcβ trisaccharide available from Stemcell Technologies (Grenoble, France).

In this context, the term “A70-A/A9” should be understood as referring to the mouse IgG1 antibody clone A70-A/A9 against Fucα1-2Galβ1-3GalNAcβ trisaccharide available from Glycotope (Berlin, Germany).

In this context, the term “A51-B/A6” should be understood as referring to the mouse IgG1 antibody clone A51-B/A6 against Fucα1-2Galβ disaccharide and capable of binding to the Fucα1-2Galβ1-3GalNAcβ trisaccharide available from Glycotope (Berlin, Germany).

In this context, the term “MBr1” should be understood as referring to the mouse IgM antibody clone MBr1 against Globo-H antigen and capable of binding to the Fucα1-2Galβ1-3GalNAcβ trisaccharide. MBr1 is available from Enzo Life Sciences (New York, USA).

These antibodies have the added utility that they are capable of binding an epitope consisting of said terminal non-reducing trisaccharide structure with high specificity and/or selectivity. In one embodiment, 17-206, anti-SSEA-5, A70-A/A9, A51-B/A6 and/or MBr1 is a xeno-free antibody.

In one embodiment, the binding agent is a lectin. Lectins occur ubiquitously in nature and typically are non-enzymatic in action and non-immune in origin. They may bind to a soluble saccharide (di- or trisaccharide) or oligosaccharide structure or to a saccharide (di- or trisaccharide) or an oligosaccharide structure that is a part of a more complex structure, such as a glycoprotein or a glycolipid. Lectins may be derived from plants, but may also have an animal origin. Known lectins isolated from plants are, for example, Con A, LCA, PSA, PCA, GNA, HPA, WGA, PWM, TPA, ECA, DSA, UEA-1, PNA, SNA and MAA. A number of lectins are readily obtainable using methods known in the art or are commercially available. Some lectins however, although capable of binding the saccharide (di- or trisaccharide) or the oligosaccharide structure, can be mitogenic or toxic.

In one embodiment, the lectin is peanut agglutinin (PNA). In this context, the term “peanut agglutinin” should be understood as referring to a Galβ1-3GalNAc specific lectin isolated from the seeds of Arachis hypogaea and available from e.g. Sigma-Aldrich (St. Louis, Mo., USA).

In one embodiment, the lectin is Bauhinea purpurea lectin (BPL), Maclura pomifera lectin (MPL), Sophora japonica lectin (SJL), Artocarpus lakoocha (Artocarpin) lectins or Abrus precatorius agglutinin (APA).

In this context, the terms “BPL”, “MPL”, “SJL”, “APA” and “Artocarpin” should be understood as referring to Galβ1-3GalNAc specific lectins that can be isolated from natural sources.

In one embodiment, the binding agent is selected from the group consisting of A68-E/E3, A68-E/A2, A68-B/A11, peanut agglutinin, BPL, MPL, SJL, APA, Artocarpin and any combination thereof.

In step b), the mixture comprising induced pluripotent stem cells and non-pluripotent cells obtainable from step a) may be contacted with the binding agent using a number of methods. For instance, the binding agent and the mixture comprising induced pluripotent stem cells and non-pluripotent cells obtainable from step a) may be incubated together in a solution.

In one embodiment, the binding agent comprises a detection-enabling molecule. Examples of detection-enabling molecules are molecules conveying affinity such as biotin or a His tag comprising at least five histidine (His) residues; molecules that have enzymatic activity such as horseradish peroxidase (HRP) or alkaline phosphatase (AP); various fluorescent molecules such as FITC, TRITC, and the Alexa and Cy dyes; gold; radioactive atoms or molecules comprising such; chemiluminescent or chromogenic molecules and the like, which molecules provide a signal for visualization or quantitation.

In one embodiment, the binding agent comprises a fluorescent molecule.

An induced pluripotent stem cell bound by the binding agent comprising a detection-enabling molecule may be detected by detecting the detection-enabling molecule.

In step c), an induced pluripotent stem cell bound by the binding agent may be selected using a number of methods. For instance, an induced pluripotent stem cell bound by a binding agent that comprises a detection-enabling molecule such as a fluorescent molecule may be selected by detecting the fluorescence emitted by the detection-enabling molecule using a fluorescent microscope.

In this context, the term “an induced pluripotent stem cell” should be understood as referring to one or more induced pluripotent stem cells and should be understood as also referring to a plurality, a colony, or a population of induced pluripotent stem cells.

In one embodiment, the term “an induced pluripotent stem cell” should be understood as referring to one induced pluripotent stem cell.

In one embodiment, the term “an induced pluripotent stem cell” should be understood as referring to at least one induced pluripotent stem cell.

In one embodiment, the term “an induced pluripotent stem cell” should be understood as referring to a plurality of induced pluripotent stem cells.

In one embodiment, the term “an induced pluripotent stem cell” should be understood as referring to a colony of induced pluripotent stem cells.

In one embodiment, the term “an induced pluripotent stem cell” should be understood as referring to a population of induced pluripotent stem cells.

In one embodiment, the term “an induced pluripotent stem cell” should be understood as referring to a monoclonal population of induced pluripotent stem cells.

In one embodiment, the term “an induced pluripotent stem cell” should be understood as referring to a heterogeneous population of induced pluripotent stem cells.

Whether one or more induced pluripotent stem cells are selected in step c) may depend on the method of selecting an induced pluripotent stem cell. Certain methods, such as many methods of cell separation including e.g. fluorescent-activated cell sorting, allow for selecting single induced pluripotent stem cells. Certain other methods, such as manual and/or mechanical selection, may only allow for selection of a colony or a population of induced pluripotent stem cells.

In one embodiment, the method comprises:

step a) of inducing non-pluripotent cells to produce a mixture comprising induced pluripotent stem cells and non-pluripotent cells;

step b) of contacting the mixture comprising induced pluripotent stem cells and non-pluripotent cells obtainable from step a) with a binding agent,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal; and

step c) of selecting one or more induced pluripotent stem cells bound by the binding agent.

In one embodiment, the method comprises:

step a) of inducing non-pluripotent cells to produce a mixture comprising induced pluripotent stem cells and non-pluripotent cells;

step b) of contacting the mixture comprising induced pluripotent stem cells and non-pluripotent cells obtainable from step a) with a binding agent,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal; and

step c) of selecting a population or a colony of induced pluripotent stem cells bound by the binding agent.

In one embodiment, an induced pluripotent stem cell bound by the binding agent is selected by visual selection.

In one embodiment, an induced pluripotent stem cell bound by the binding agent is selected and isolated. An induced pluripotent stem cell may be selected and isolated simultaneously, or an induced pluripotent stem cell may be selected first and isolated only after a time period or at a later stage or step.

In one embodiment, an induced pluripotent stem cell bound by the binding agent is selected and isolated using manual isolation. Manual isolation may include e.g. manually detaching and picking an induced pluripotent stem cell or a colony of induced pluripotent stem cells.

In one embodiment, an induced pluripotent stem cell bound by the binding agent is selected and isolated using mechanical isolation.

In one embodiment, an induced pluripotent stem cell bound by the binding agent is selected and isolated using a method of cell separation.

Various methods of cell separation are known in the art, e.g. separation by non-magnetic techniques; affinity separation such as affinity chromatography; separation by magnetic techniques, e.g. immunomagnetic beads or magnetic cell sorting; flow cytometry, e.g. fluorescent-activated cell sorting (FACS); or dielectrophoresis.

In one embodiment, the method of cell separation is fluorescent-activated cell sorting, magnetic-activated cell sorting or affinity chromatography.

In one embodiment, the method of cell separation is fluorescent-activated cell sorting (FACS).

In order to allow for selecting and isolating an induced pluripotent stem cell bound by the binding agent, the binding agent may comprise a detection-enabling molecule. For instance, in embodiments wherein the method of cell separation is fluorescent-activated cell sorting, the binding agent may comprise a fluorescent molecule.

In one embodiment, step b) further comprises contacting the mixture comprising induced pluripotent stem cells and non-pluripotent cells obtainable from step a) with a secondary binding agent capable of binding a secondary epitope.

Contacting the mixture comprising induced pluripotent stem cells and non-pluripotent cells obtainable from step a) with a binding agent capable of binding an epitope consisting of non-reducing terminal saccharide structure according to the formula (Fucα1-2)ₐGalβ1-3HexNAcβ, wherein a=0 or 1 and Hex is either Glc or Gal; and with a secondary binding agent capable of binding a secondary epitope may be done simultaneously or sequentially. When done sequentially, the order in which the binding agent and the secondary binding agent are contacted with the mixture comprising induced pluripotent stem cells and non-pluripotent cells obtainable from step a) may be chosen e.g. depending on the secondary epitope, the secondary binding agent and other considerations.

In one embodiment, step c) comprises selecting an induced pluripotent stem cell bound by the binding agent and by a secondary binding agent capable of binding a secondary epitope.

Embodiments in which a secondary binding agent is used have the added utility that they allow for selecting an induced pluripotent stem cell comprising two or more epitopes, which may improve the selectivity and efficiency of the method.

The secondary binding agent may be an antibody or a lectin.

In one embodiment, the secondary binding agent is a xeno-free secondary binding agent.

In one embodiment, the secondary epitope is an epitope enriched in induced pluripotent stem cells.

In one embodiment, the secondary epitope is selected from the group consisting of the non-reducing terminal saccharide structure according to formula (Fucα1-2)ₐGalβ1-3HexNAcβ, wherein a=0 or 1 and Hex is either Glc or Gal, K21, Tra-1-60, Tra-1-81, Tra-2-54, SSEA-3, SSEA-4, H type 1, Lewis y, alkaline phosphatase, CD9, CD24, CD29, CD30, CD44, CD49c, CD49f, CD50, CD51/61, CD56, CD57, CD58, CD71, CD73, CD98, CD105, CD117, CD133, CD140a, CD146, CD193, CD196, CD271, CD309, CD326, CD338, GCTM-2, TG30, and TG343.

In one embodiment, the secondary binding agent is a binding agent capable of binding the secondary epitope Tra-1-60.

In one embodiment, the secondary binding agent is the antibody Tra-1-60. In one embodiment, Tra-1-60 is a xeno-free antibody.

In this context, the term “a secondary binding agent” should be understood as referring to one or more secondary binding agents. For instance, the term “a secondary binding agent” may refer to two to three different secondary binding agents. In one embodiment, a secondary binding agent is a xeno-free agent. In one embodiment, the secondary binding agent is a xeno-free antibody capable of binding a secondary epitope selected from the group consisting of (Fucα1-2)ₐGalβ1-3HexNAcβ, wherein a=0 or 1 and Hex is either Glc or Gal, K21, Tra-1-81, Tra-2-54, SSEA-3, SSEA-4, H type 1, Lewis y, alkaline phosphatase, CD9, CD24, CD29, CD30, CD44, CD49c, CD49f, CD50, CD51/61, CD56, CD57, CD58, CD71, CD73, CD98, CD105, CD117, CD133, CD140a, CD146, CD193, CD196, CD271, CD309, CD326, CD338, GCTM-2, TG30, and TG343.

In one embodiment, step c) comprises selecting an induced pluripotent stem cell bound by the binding agent and not bound by a secondary binding agent capable of binding a secondary epitope.

In one embodiment, the secondary epitope is an epitope enriched in non-pluripotent cells.

In one embodiment, the secondary epitope is selected from the group consisting of CD7, CD13, CD26, CD31, CD34, CD45, CD46, SSEA-1, GD2, GD3, and GT3. In one embodiment, the secondary binding agent is a xeno-free antibody capable of binding a secondary epitope selected from the group consisting of CD7, CD13, CD26, CD31, CD34, CD45, CD46, SSEA-1, GD2, GD3, and GT3.

Embodiments in which step c) comprises selecting an induced pluripotent stem cell not bound by a secondary binding agent capable of binding a secondary epitope have the added utility that they allow for excluding from selection an induced pluripotent stem cell comprising a secondary epitope that is associated with non-pluripotent cells, for instance induced cells that are only partially reprogrammed; this may improve the selectivity and efficiency of the method. For instance, the epitope CD13 is enriched in human adult fibroblast cells; thus selecting an induced pluripotent stem cell not bound by a binding agent capable of binding the epitope CD13 may improve the selectivity of the method. Methods of cell separation such as fluorescent-activated cell sorting may allow simultaneous selection of induced pluripotent stem cells based on binding to a number of, for instance 2 to 16, binding agents and secondary binding agents.

Secondary binding agents, e.g. antibodies against secondary epitopes such as Tra-1-60 or CD13, are commercially available.

In one embodiment, the secondary binding agent further comprises a detection-enabling molecule.

In one embodiment, the secondary binding agent comprises a fluorescent molecule.

In one embodiment, a population of cells bound by the binding agent is selected in step c). While the method according to one or more embodiments of the invention is very efficient, and the population bound by the binding agent mainly comprises induced pluripotent stem cells, the population of cells bound by the binding agent selected in step c) may still comprise some non-pluripotent cells.

In one embodiment, at least 80%, or at least 90%, or at least 95%, or at least 97%, or at least 98%, or at least 99%, or at least 99.5%, or 100% of the cells of the population selected in step c) are induced pluripotent stem cells. The proportion of induced pluripotent stem cells of the cells of the population selected in step c) may be determined e.g. by selecting a number (e.g. at least 100, 500, or 1000) cells in step c) and cultivating them, for instance in individual wells of a multiwell plate. After a time period, the cells may be observed and the proportion of the induced pluripotent stem cells of the cells of the population selected in step c) determined. The time period may be e.g. a time period sufficient for the cells to form colonies. Cells which have produced morphologically stable colonies that are free of non-pluripotent and/or differentiated cells may be considered to be induced pluripotent stem cells. Cells which have produced morphologically non-stable colonies and/or colonies which contain non-pluripotent, incompletely reprogrammed cells and/or differentiated cells may not be considered to be induced pluripotent stem cells. Induced pluripotent stem cells typically produce morphologically stable colonies that are free of differentiated cells after two passages. The induced pluripotent stem cells may be further verified e.g. by assaying expression of stem cell markers.

In one embodiment, the induced pluripotent stem cell expresses at least three, or at least four, or at least five, or at least six, or at least seven, or all of the stem cell markers selected from the group consisting of Tra-1-60, Tra-1-81, SSEA-3, H type 1, OCT-4, NANOG, SOX2 and TGFβ1.

In one embodiment, at least 80%, or at least 90%, or at least 95%, or at least 97%, or at least 98%, or at least 99%, or at least 99.5%, or 100% of the cells of the population selected in step c) are stable induced pluripotent stem cells.

In one embodiment, the proportion of induced pluripotent stem cells in the population of cells bound by the binding agent selected in step c) is at least 1.5-fold higher, or at least 2-fold higher, or at least 2.5-fold higher than the proportion of induced pluripotent stem cells in the population of cells generated by a comparable method in which the antibody Tra-1-60 is used as a binding agent.

In other words, the proportion of induced pluripotent stem cells in the population of cells bound by the binding agent selected in step c) is at least 1.5-fold higher, or at least 2-fold higher, or at least 2.5-fold higher than the proportion of induced pluripotent stem cells in the population of cells generated by a comparable method for generating induced pluripotent stem cells comprising

step a′) of inducing non-pluripotent cells to produce a mixture comprising induced pluripotent stem cells and non-pluripotent cells;

step b′) of contacting the mixture comprising induced pluripotent stem cells and non-pluripotent cells obtainable from step a) with the antibody Tra-1-60; and

step c′) of selecting an induced pluripotent stem cell bound by the antibody Tra-1-60.

In one embodiment, the method further comprises:

step d) of cultivating the induced pluripotent stem cell obtainable from step c).

In this context, the term “the induced pluripotent stem cell obtainable from step c)” should be understood as referring to one or more induced pluripotent stem cells obtainable from step c) and should be understood as also referring to a plurality, a colony, or a population of induced pluripotent stem cells obtainable from step c).

In one embodiment, the term “the induced pluripotent stem cell obtainable from step c)” should be understood as referring to one induced pluripotent stem cell obtainable from step c). This embodiment has the added utility that a monoclonal population of induced pluripotent stem cells may be obtained in step d).

In step d) and/or step a), the induced pluripotent stem cell obtainable from step c) and/or the mixture comprising induced pluripotent stem cells and non-pluripotent cells obtained in step a) may be expanded.

In one embodiment, the induced pluripotent stem cell obtainable from step c) and/or the mixture comprising induced pluripotent stem cells and non-pluripotent cells obtained in step a) is cultivated at clonal density in step d) and/or in step a). This embodiment has the added utility that a monoclonal colony or population of induced pluripotent stem cells may be obtained in step d) and/or step a).

In this context, the term “clonal density” should be understood as referring to a density at which induced pluripotent stem cells are capable of forming individual colonies.

In one embodiment, a plurality, a colony or a population of induced pluripotent stem cells obtainable from step c) and/or the mixture comprising induced pluripotent stem cells and non-pluripotent cells obtained in step a) are cultivated in step d) and/or in step a) at a density higher than clonal density. This embodiment has the added utility that cultivating the induced pluripotent stem cells obtainable from step c) and/or the mixture comprising induced pluripotent stem cells and non-pluripotent cells obtained in step a) at a density higher than clonal density may improve the survival rate of the induced pluripotent stem cells obtainable from step c) and/or the mixture comprising induced pluripotent stem cells and non-pluripotent cells obtained in step a). In this embodiment, a monoclonal colony or population of induced pluripotent stem cells may be obtained in a subsequent step by e.g isolating one or more induced pluripotent stem cells obtainable from step d) and cultivating in a secondary cultivation at a clonal density.

During cultivation in step d) and/or in step a), the induced pluripotent stem cell obtainable from step c) and/or the mixture comprising induced pluripotent stem cells and non-pluripotent cells obtained in step a) is seeded or passaged to a cultivation system, which may include, for instance, a cultivation vessel, a matrix or substrate and a culture medium. Exemplary cultivation vessels include, but are not limited to, a 6 well plate, a 12 well plate, a 24 well plate, a 48 well plate, a 96 well plate, and a 384 well plate. The starting point of the cultivation may thus be defined as the time point at which the induced pluripotent stem cell obtainable from step c) and/or the mixture comprising induced pluripotent stem cells and non-pluripotent cells obtained in step a) is contacted with the cultivation system.

In one embodiment, the induced pluripotent stem cell is selected or sorted in step c) directly to a multiwell plate. In such an embodiment, the induced pluripotent stem cells may be cultivated and optionally also differentiated in the well to which is has been selected or sorted.

In one embodiment, the induced pluripotent stem cell is selected or sorted directly to a multiwell plate, such as a 96 or 384 well plate, at a density of one induced pluripotent stem cell per one well or 2-10 induced pluripotent stem cells per one well. In embodiments in which the induced pluripotent stem cell is selected or sorted directly to a multiwell plate at a density of one induced pluripotent stem cell per one well, a monoclonal population or colony may be derived from the induced pluripotent stem cell.

In one embodiment, the induced pluripotent stem cells are selected or sorted directly to a multiwell plate, such as a 24 well plate, at a density of 100-500 induced pluripotent stem cells per one well, or about 500, or about 1000-2000, or about 2000-5000 induced pluripotent stem cells per one well. In such an embodiment, a population of induced pluripotent stem cells may be obtained in a time period shorter than if the induced pluripotent stem cells were selected or sorted at a density of one induced pluripotent stem cell per one well.

In one embodiment, the induced pluripotent stem cell may be selected or sorted directly to a frequency of one induced pluripotent stem cell per a well of a 6 well plate, a 12 well plate, a 24 well plate, a 48 well plate, a 96 well plate, or a 384 well plate. In one embodiment, the induced pluripotent stem cell may be selected or sorted directly to a frequency of 2-10 iPS cells per a well of a 6 well plate, a 12 well plate, a 24 well plate, a 48 well plate, a 96 well plate, or a 384 well plate. Single-cell dissociation of induced pluripotent stem cells followed by single cell passaging may be used in the present methods with several advantages, such as facilitating cell expansion, cell sorting, and defined seeding for differentiation and enabling automatization of culture procedures and clonal expansion.

Culture medium may be added to the cultivation (cultivation system) or replaced as required. Further, additional substances may be added to culture medium or cultivation system as desired. Induced pluripotent stem cells are grown and maintained at an appropriate temperature and gas mixture. Typically, 37° C. and 5% CO₂ are used for mammalian cells. Atmospheric O₂ pressure can be used, or alternatively lowered O₂ pressure (hypoxia) for optimized stem cell growth conditions. However, the exact conditions may depend e.g. on the desired outcome of the cultivation. For example, if induced pluripotent stem cells are generated by transfecting mRNAs into non-pluripotent cells, optimal oxygen levels may be in range of 3 to 5%.

Induced pluripotent stem cells may be selected or sorted directly to suspension culture. Induced pluripotent stem cells in suspension culture may be single cell or induced pluripotent stem cell aggregate culture. Single induced pluripotent stem cells may be derived directly from selection step c) of the present invention or induced pluripotent stem cell aggregates dissociated by mechanical (cell scraper, trituration through a narrow bore pipette, fine needle aspiration, vortex disaggregation and forced filtration through a fine nylon or stainless steel mesh) or chemical means (trypsin, collagenase, EDTA, Acutase and the like) or a combination of both. Induced pluripotent stem cell aggregates can be grown in 3D culture substrate particles such as microcarriers, optionally coated with e.g. Matrigel or a carbohydrate-binding protein capable of binding the non-reducing terminal oligosaccharide structure according to the formula

(Fucα1-2)ₙGalβ1-4GlcNAc, wherein n=0 or 1. In one embodiment, the carbohydrate-binding protein is ECA. In one embodiment, single cell and iPS cell aggregates are contacted with the carbohydrate-binding protein and a Rho-associated kinase inhibitor such as pinacidil or Y-27632.

In one embodiment, the induced pluripotent stem cell obtainable from step c) and/or the mixture comprising induced pluripotent stem cells and non-pluripotent cells obtained in step a) is cultivated on a suitable matrix in step d) and/or in step a).

A suitable matrix for cultivating the induced pluripotent stem cell obtainable from step c) in step d) and/or the mixture comprising induced pluripotent stem cells and non-pluripotent cells obtained in step a) may be e.g. a feeder cell layer comprising mouse embryonic fibroblasts. Another suitable matrix may be an extracellular matrix extracted from a mouse sarcoma sold under the trademark Matrigel™ (BD Biosciences, US), herein referred to as Matrigel. Another suitable matrix may be truncated recombinant human vitronectin, recombinant laminin(s) (e.g. provided by BioLamina AB, for example LN-521), StemAdhere™, Synthemax™-R, StemXVivo™, collagen, fibronectin, or modified Matrigel such as Geltrex® (e.g. from Life Technologies). During the cultivation, the induced pluripotent stem cell obtainable from step c) and/or the mixture comprising induced pluripotent stem cells and non-pluripotent cells obtained in step a) typically adheres to a matrix or a substrate included in the cultivation system, such as a feeder cell layer or Matrigel. This adherence depends on the interaction of the induced pluripotent stem cell obtainable from step c) and/or the mixture comprising induced pluripotent stem cells and non-pluripotent cells obtained in step a) with the components of the matrix or substrate.

In one embodiment, the induced pluripotent stem cell obtainable from step c) and/or the mixture comprising induced pluripotent stem cells and non-pluripotent cells obtained in step a) are cultivated in step d) and/or in step a) in a culture essentially free of feeder cells.

In one embodiment, the induced pluripotent stem cell obtainable from step c) and/or the mixture comprising induced pluripotent stem cells and non-pluripotent cells obtained in step a) are cultivated in step d) and/or in step a) in a culture free of feeder cells.

A “feeder cell” is a cell that grows in vitro and that may be co-cultured in step d) and/or step a) with an induced pluripotent stem cell obtainable from step c) and/or the mixture comprising induced pluripotent stem cells and non-pluripotent cells obtained in step a). The term “essentially free of a feeder cell” refers to tissue culture conditions that do not contain feeder cells or a feeder layer.

In one embodiment, the induced pluripotent stem cell obtainable from step c) and/or the mixture comprising induced pluripotent stem cells and non-pluripotent cells obtained in step a) are cultivated in step d) and/or in step a) in xeno-free culture medium. In this context “xeno-free” should be understood as referring to that all components, for example, of a medium do not contain any animal-derived components. In certain embodiments, all components of a culture medium are chemically defined, human recombinant proteins produced without any animal-derived molecules or serum components.

Xeno-free antibody means an antibody which is produced in defined media, xeno-free conditions. If the antibody is produced in a cell line, the cell line is cultured in defined media, xeno-free conditions in cell line's entire existence from cell line's initial generation to antibody transfection and antibody isolation. Particularly, the cell line has not been in contact with bovine or equine derived components or sera. Further, it is understood that, for example, an anti-PLN produced in, for example, CHO cells in a xeno-free, defined culture medium is a xeno-free anti-PLN antibody. In one embodiment, the induced pluripotent stem cell obtainable from step c) and/or the mixture comprising induced pluripotent stem cells and non-pluripotent cells obtained in step a) are cultivated in step d) and/or in step a) in a defined medium which is a non-conditioned medium, i.e. a medium that is not obtained from a feeder cell.

In one embodiment, cells in any or in all steps of steps a), b), c), and d) are cultured in a defined and/or xeno-free culture medium.

For generating induced pluripotent stem cells from non-pluripotent cells, various optimized processes, culture media, and matrices have been developed. See, for example, Chen et al. (2014), Human pluripotent stem cell culture: considerations for maintenance, expansion, and therapeutics. Cell Stem Cell, 14:13-26.

In one embodiment, the culture medium is E6 or E8 medium or Knockout serum-free medium available from Life Technologies, NutriStem™ (from StemGent), DEF-CS 500 (from Cellectis), or mTeSR-1 or mTeSR-2 medium (from Stemcell Technologies).

In one embodiment, the induced pluripotent stem cell obtainable from step c) are cultivated in step d) to form a colony (one or more colonies) comprising induced pluripotent stem cells and optionally passaging the induced pluripotent stem cells one or more times.

In one embodiment, the method further comprises step e) of differentiating the induced pluripotent stem cells to a differentiated cell type or a differentiated lineage.

In one embodiment, the method comprises:

step a) of inducing non-pluripotent cells to produce a mixture comprising induced pluripotent stem cells and non-pluripotent cells and cultivating the mixture comprising induced pluripotent stem cells and non-pluripotent cells for a time period, such as a time period that is shorter than a time period required for the mixture comprising induced pluripotent stem cells and non-pluripotent cells to form colonies;

step b) of contacting the mixture comprising induced pluripotent stem cells and non-pluripotent cells obtainable from step a) with a binding agent,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal,

and with a secondary binding agent capable of binding a secondary epitope, such as the antibody Tra-1-60;

step c) of selecting an induced pluripotent stem cell bound by the binding agent and by the secondary binding agent capable of binding the secondary epitope;

step d) of cultivating the induced pluripotent stem cell obtainable from step c) to form a colony comprising induced pluripotent stem cells and optionally passaging the induced pluripotent stem cells one or more times; and

step e) of differentiating the induced pluripotent stem cell to a differentiated cell type or a differentiated lineage.

In one embodiment, in step d), the induced pluripotent stem cell may be cultivated at a density in which the induced pluripotent stem cell forms an individual colony. For instance, individual (single) induced pluripotent stem cells may be cultivated in individual wells of a culture vessel.

In one embodiment, the induced pluripotent stem cell is cultivated and differentiated to a differentiated cell type or a differentiated lineage in the same culture vessel. In other words, the (one or more) induced pluripotent stem cell is not passaged to another culture vessel prior to differentiating.

In one embodiment, the induced pluripotent stem cell obtainable from step c) and/or the mixture comprising induced pluripotent stem cells and non-pluripotent cells obtained in step a) is contacted with a carbohydrate-binding protein during the cultivation in step d) and/or in step a),

wherein the carbohydrate-binding protein is capable of binding the non-reducing terminal oligosaccharide structure according to the formula

(Fucα1-2)ₙGalβ1-4GlcNAc,

wherein n=0 or 1.

This embodiment has the added utility that the carbohydrate-binding protein ensures efficient attachment and is thus a potent simple defined matrix for the induced pluripotent stem cell obtainable from step c) and/or the mixture comprising induced pluripotent stem cells and non-pluripotent cells obtained in step a). Contacting the induced pluripotent stem cell obtainable from step c) and/or the mixture comprising induced pluripotent stem cells and non-pluripotent cells obtained in step a) with the carbohydrate-binding protein may also improve the survival of the induced pluripotent stem cell obtainable from step c) and/or the mixture comprising induced pluripotent stem cells and non-pluripotent cells obtained in step a). Thus the carbohydrate-binding protein may improve the cultivation efficiency of induced pluripotent stem cells obtainable from step c) and/or the mixture comprising induced pluripotent stem cells and non-pluripotent cells obtained in step a) at a clonal density.

In this context, the abbreviation “Fuc” should be understood as L-fucose; “Neu5Ac” should be understood as N-acetylneuraminic acid; “Neu5Gc” should be understood as N-glycolylneuraminic acid; and “GlcNAc” and “N-acetylglucosamine” should be understood as 2-acetamido-2-deoxy-D-glucose; and all monosaccharides are in pyranose form.

In one embodiment, the non-reducing end terminal oligosaccharide structure is not substituted by any other monosaccharide residue or any other substituent on any other positions than at the reducing end of the oligosaccharide structure.

In this context, the term “carbohydrate-binding protein” should be understood as referring to any protein, provided it is capable of binding the non-reducing terminal oligosaccharide structure according to the formula

(Fucα1-2)ₙGalβ1-4GlcNAc,

wherein n=0 or 1.

In this context, the term “a carbohydrate-binding protein” should be understood as referring to at least one carbohydrate-binding protein.

In one embodiment, the carbohydrate-binding protein is capable of binding the non-reducing terminal oligosaccharide structure according to the formula Fucα1-2Galβ1-4GlcNAc.

In one embodiment, the carbohydrate-binding protein is capable of binding the non-reducing terminal oligosaccharide structure according to the formula Galβ1-4GlcNAc.

In one embodiment, the carbohydrate-binding protein is capable of binding the non-reducing terminal oligosaccharide structure according to both the formulas Galβ1-4GlcNAc and Fucα1-2Galβ1-4GlcNAc.

In one embodiment, the carbohydrate-binding protein is a lectin, an antibody or a carbohydrate-modifying protein, or a modification or a fragment thereof.

In one embodiment, the carbohydrate-binding protein is a lectin.

In one embodiment, the carbohydrate-binding protein has essentially the binding specificity of ECA, UEA-I, DSA, RCA, galectin, an antibody or a carbohydrate-modifying protein, or a modification or a fragment thereof, the binding specificity being for the non-reducing terminal oligosaccharide structure Galβ1-4GlcNAc and/or Fucα1-2Galβ1-4GlcNAc.

In this context, the abbreviation “ECA” should be understood as referring to lectin (agglutinin) from Erythrina cristagalli (also called ESL) and homologous lectins from Erythrina species e.g. as defined in Bhattacharyya, L., et al. (1989, Glycoconj. J. 6:141-50), WO2010004096, and WO2008087257, such as lectins of E. corallodendron, E. flabelliformis, and E. indica. ECA is capable of binding to N-acetyllactosamine (type 2 chain) glycoconjugates, a common structure in glycans of undifferentiated stem cells.

In one embodiment, the abbreviation “ECA” should be understood as also referring to a carbohydrate-binding protein that has essentially the binding specificity of ECA, i.e. the binding specificity for the non-reducing terminal oligosaccharide structure Galβ1-4GlcNAc and/or Fucα1-2Galβ1-4GlcNAc.

In this context, the abbreviation “UEA-1” should be understood as referring to a lectin (agglutinin-I) from Ulex europeaus.

In this context, the abbreviation “DSA” should be understood as referring to a lectin from Datura stramonium.

In this context, the abbreviation “RCA” should be understood as referring to a non-toxic lectin domain of an agglutinin from Ricinus communis.

In this context, the term “galectin” should be understood as referring to a family of animal lectins capable of binding beta-galactoside, preferably selected from the group of galectins 1-15 encoded by genes named LGALS.

In one embodiment, galectin is mammalian or human galectin-1. This embodiment has the added utility that mammalian or human galectin-1 has good binding specificity and can support the attachment and undifferentiated proliferation of the induced pluripotent stem cell obtainable from step c) and/or the mixture comprising induced pluripotent stem cells and non-pluripotent cells obtained in step a).

In one embodiment, the carbohydrate-binding protein is ECA, UEA-1, DSA, RCA, galectin, or a modification or a fragment thereof.

In one embodiment, the carbohydrate-binding protein is ECA. This embodiment has the added utility that ECA binds highly specifically to the non-reducing terminal oligosaccharide structure as defined above. ECA can support the attachment and undifferentiated proliferation of the induced pluripotent stem cell obtainable from step c) and/or the mixture comprising induced pluripotent stem cells and non-pluripotent cells obtained in step a). ECA is also a small-sized protein that can easily be produced recombinantly and thus is suitable for GMP use. Further, ECA exhibits low mitogenicity and toxicity.

In one embodiment, the carbohydrate-binding protein is an antibody or any modification or a fragment thereof. As an example only, the carbohydrate-binding protein may be an scFv, a single domain antibody, an Fv, a VHH antibody, a diabody, a tandem diabody, a Fab, a Fab′, or a F(ab)2, provided it binds said non-reducing terminal oligosaccharide structure. Furthermore, the antibody, modification or a fragment thereof may be present in monovalent monospecific, multivalent monospecific, bivalent monospecific, or multivalent multispecific forms. Methods for producing and screening antibodies are well known in the art.

In one embodiment, the antibody or a modification or a fragment thereof is, or comprises the complementary-determining regions of, an antibody specific for Fucα1-2Galβ1-4GlcNAc (H type II) or Galβ1-4GlcNAc as defined in WO2008087259, which document is herein incorporated in its entirety.

In one embodiment, the carbohydrate-binding protein is a carbohydrate-modifying protein. In one embodiment, the carbohydrate-modifying protein is selected from the group of glycosyltransferases and glycosidases specific for Fucα1-2Galβ1-4GlcNAc or Galβ1-4GlcNAc including ST3GalII, ST3GalIV, ST6Gal, ST6GalII, FucT-IV, FucT-IX, FucT-VI, blood group A GalNAc-transferase, blood group B Gal-transferase, α1,2-fucosidase and β1,4-galactosidase.

In this context, the term “fragment” should be understood as referring to a portion of the binding agent or the carbohydrate-binding protein that is capable of binding the epitope consisting of the non-reducing terminal disaccharide structure or the non-reducing terminal oligosaccharide structure.

In one embodiment, the carbohydrate-binding protein exhibits selective binding to the non-reducing terminal oligosaccharide structures Fucα1-2Galβ1-4GlcNAc and/or Galβ1-4GlcNAc over other common structures selected from the group of Galβ1-4(Fucα1-3)GlcNAc, Fucα1-2Galβ1-4(Fucα1-3)GlcNAc, Neu5Acα2-3Galβ1-4GlcNAc and Neu5Acα2-6Galβ1-4GlcNAc. In one embodiment, the galectin may exhibit additional binding to Neu5Acα2-3Galβ1-4GlcNAc or Neu5Acα2-6Galβ1-4GlcNAc. In one embodiment, the selective binding means at least 10, 20, 25, 50, 100, or 1000-fold binding affinity compared to the other common structures.

In one embodiment, the carbohydrate-binding protein may be contacted with the induced pluripotent cell obtainable from step c) and/or the mixture comprising induced pluripotent stem cells and non-pluripotent cells obtained in step a) e.g. as a coating on the surface of a culture vessel, such as a culture dish or plate. In one embodiment, the carbohydrate-binding protein may be contacted with the induced pluripotent cell obtainable from step c) in immobilized form immobilized to a three-dimensional structure, such as a gel. Alternatively, the carbohydrate-binding protein may be contacted with the induced pluripotent cell obtainable from step c) and/or the mixture comprising induced pluripotent stem cells and non-pluripotent cells obtained in step a) e.g. as immobilized on the surface of particles such as microcarriers contained in a culture system or vessel.

In one embodiment, the induced pluripotent stem cell obtainable from step c) and/or the mixture comprising induced pluripotent stem cells and non-pluripotent cells obtained in step a) is contacted with the carbohydrate-binding protein as a matrix or comprised in a matrix.

In this context, the term “matrix” should be understood as referring to a substrate to which the induced pluripotent cell obtainable from step c) and/or the mixture comprising induced pluripotent stem cells and non-pluripotent cells obtained in step a) can adhere. The matrix may be provided e.g. as a coating on the surface of a culture vessel, such as a culture dish or plate. Alternatively, the matrix may be provided in immobilized form immobilized to a three-dimensional structure, such as a gel. Alternatively, the matrix may be provided e.g. as a coating on the surface of particles, such as microcarriers, contained in a culture system or vessel. Alternatively, the carbohydrate-binding protein may be provided in covalently conjugated form immobilized to the matrix, vessel, three-dimensional structure or particle. In one embodiment, the carbohydrate-binding protein may be provided in immobilized or covalently conjugated form as defined in the publications WO2010004096 and WO2008087257.

The carbohydrate-binding protein should be present in an effective amount. The effective amount may depend on, amongst other things, the particular carbohydrate-binding protein, the induced pluripotent stem cell obtainable from step c) and/or the mixture comprising induced pluripotent stem cells and non-pluripotent cells obtained in step a), the culture system used and whether the carbohydrate-binding protein is immobilized or provided as a coating. In one embodiment, the amount of the carbohydrate-binding protein used in a solution is about 0.1-500 μg/ml, or about 5-200 μg/ml, or about 10-150 μg/ml. The amount of the carbohydrate-binding protein for immobilization is about 0.001-50 μg/cm², or about 0.01-50 μg/cm², or about 0.1-30 μg/cm² for a carbohydrate-binding protein with a molecular weight of about 50 kDa, or a corresponding molar density per surface area. In one embodiment, about 1-50 μg/cm², or about 5-40 μg/cm², or about 10-40 μg/cm² of the carbohydrate-binding protein is used in a solution to coat a plastic cell culture surface. In one embodiment, the concentration of the coating solution is about 50-200 μg/ml for a carbohydrate-binding protein with a molecular weight of about 50 kDa, or in a corresponding molar concentration.

In one embodiment, the induced pluripotent stem cell obtainable from step c) and/or the mixture comprising induced pluripotent stem cells and non-pluripotent cells obtained in step a) is contacted with a Rho-associated kinase inhibitor at one or more time intervals during the cultivation in step d) and/or in step a). The induced pluripotent stem cell obtainable from step c) and/or the mixture comprising induced pluripotent stem cells and non-pluripotent cells obtained in step a) may be contacted with a Rho-associated kinase inhibitor at one or more time intervals during the cultivation in step d) and/or in step a) and with any suitable matrix. The matrix may be any matrix described in this document. The matrix may also be selected e.g. from the group consisting of Matrigel, truncated recombinant human vitronectin, recombinant laminin(s) (e.g. provided by BioLamina AB, for example LN-521), StemAdhere™, Synthemax™-R, StemXVivo™, collagen, fibronectin, and modified Matrigel such as Geltrex®.

In one embodiment, the induced pluripotent stem cell obtainable from step c) and/or the mixture comprising induced pluripotent stem cells and non-pluripotent cells obtained in step a) is contacted with the carbohydrate-binding protein and a Rho-associated kinase inhibitor simultaneously at one or more time intervals during the cultivation in step d) and/or in step a). This embodiment has the added utility that a Rho-associated kinase inhibitor and the carbohydrate-binding protein act synergistically to promote significantly higher cell viability after dissociation compared to for example Matrigel. This embodiment also provides for rapid, undifferentiated growth of induced pluripotent stem cells and thus for very high growth and expansion rates of induced pluripotent stem cells, when compared with traditional cultivation methods involving e.g. the use of Matrigel or a feeder cell layer. This embodiment further allows for non-colony growth of induced pluripotent stem cells. In this context, the term “non-colony growth” should be understood as referring to growth as an essentially continuous layer without colony boundaries, in other words limitless growth, with an ability to essentially fill the available surface.

In this context, the term “Rho-associated kinase inhibitor” should be understood as referring to any molecule capable of selectively inhibiting Rho-associated protein kinase. Rho-associated protein kinase is a kinase belonging to the AGC (PKA/PKG/PKC) family of serine-threonine kinases. It is mainly involved in regulating the shape and movement of cells by acting on the cytoskeleton. Rho-associated kinases occur in a number of species, including human, rat, mouse, cow, and zebrafish, only to mention a few. A number of Rho-associated kinase inhibitors are known, including pinacidil, Y-27632, Fasudil (also known as HA1077), Thiazovivin, N-hydroxyfasudil, (S)-(+)-2-methyl-1-[(4-methyl-5-isoquinolinyl)sulfonyl]homo-piperazine, N-(4-pyridyl)-N′-(2,4,6-trichlorophenyl) urea, 3-(4-pyridyl)-1H-indole, glycyl(S)-(+)-2-methyl-4-glycyl-1-(4-methylisoquinolinyl-5-sulfonyl)homo-piperazine, azabenzimidazoleaminofurazan, 4-(1-amino-alkyl)-N-(4-pyridyl)cyclo-hexane-carboxamide, and Rhostatin, and are also commercially available. In this context, the term “a Rho-associated kinase inhibitor” should also be understood as referring to at least one Rho-associated kinase inhibitor.

In one embodiment, the Rho-associated kinase inhibitor is selected from the group consisting of pinacidil, Y-27632, Fasudil, Thiazovivin, N-hydroxyfasudil, (S)-(+)-2-methyl-1-[(4-methyl-5-isoquinolinyl)sulfonyl]homopiperazine, N-(4-pyridyl)-N′-(2,4,6-trichlorophenyl) urea, 3-(4-pyridyl)-1H-indole, glycyl(S)-(+)-2-methyl-4-glycyl-1-(4-methylisoquinolinyl-5-sulfonyl)homopiperazine, azabenzimidazoleaminofurazan, 4-(1-amino-alkyl)-N-(4-pyridyl)cyclohexane-carboxamide, Rhostatin, and any combination thereof.

In one embodiment, the Rho-associated kinase inhibitor is pinacidil or Y-27632.

In one embodiment, the concentration of pinacidil is between 1-1000 μM, between 10-500 μM, between 50-200 μM, or about 100 μM. In one embodiment, the concentration of Y-27632 is between 0.1-100 μM, between 1-50 μM, between 5-20 μM, or about 10 μM.

In one embodiment, the Rho-associated kinase inhibitor may be contacted with the induced pluripotent cell obtainable from step c) and/or the mixture comprising induced pluripotent stem cells and non-pluripotent cells obtained in step a) in a solubilized form, e.g. included in a culture medium or added in a culture medium. In another embodiment, the Rho-associated kinase inhibitor may be contacted with the induced pluripotent cell obtainable from step c) and/or the mixture comprising induced pluripotent stem cells and non-pluripotent cells obtained in step a) in an immobilized form, e.g. as a component of the coating on the surface of a culture vessel, such as a culture dish or plate, or of matrix such as a three-dimensional structure, e.g. a gel.

In some embodiments, the medium used in the method according to one or more embodiments of the present invention may already contain the Rho-associated kinase inhibitor. Alternatively, the method according to one or more embodiments of the present invention may comprise adding the Rho-associated kinase inhibitor to the medium during cultivation in step d) and/or in step a). The concentration of the Rho-associated kinase inhibitor in the medium is not particularly limited provided it can achieve the desired effects such as an improved survival rate of the induced pluripotent stem cell obtainable from step c). A Rho-associated kinase inhibitor, e.g. pinacidil, may be used at an effective concentration of at least or about 0.02, 0.05, 0.1, 0.2, 0.5, 1, 2, 3, 4, 5, 10, 15, 20, 25, 30, 35, 40, 45, 50, 100, 150, 200, 500 to about 1000 μM, or any range derivable therein. These amounts may refer to an amount of a Rho-associated kinase inhibitor individually or in combination with one or more Rho-associated kinase inhibitors.

The time interval for contacting the induced pluripotent cell obtainable from step c) and/or the mixture comprising induced pluripotent stem cells and non-pluripotent cells obtained in step a) with the Rho-associated kinase inhibitor is not limited as long as it is a time interval for which the desired effects such as the improved survival rate of induced pluripotent stem cells can be achieved. For example, the time interval for contacting the induced pluripotent stem cell obtainable from step c) and/or the mixture comprising induced pluripotent stem cells and non-pluripotent cells obtained in step a) with the Rho-associated kinase inhibitor may be at least or about 1, 2, 5, 10, 15, 20, 25, 30 minutes to several hours (e.g., at least or about one hour, two hours, three hours, four hours, five hours, six hours, eight hours, 12 hours, 16 hours, 24 hours, 36 hours, 48 hours, or any range derivable therefrom) before dissociation. After dissociation, the induced pluripotent cell obtainable from step c) and/or the mixture comprising induced pluripotent stem cells and non-pluripotent cells obtained in step a) may be contacted with the Rho-associated kinase inhibitor for, for example, at least or about 0.1, 0.2, 0.5, 1, 2, 3, 4, 5, 6, 7, 8, 9, 10, 11, 12, 16, 24, 48 hours or more to achieve the desired effects.

In certain embodiments, the induced pluripotent stem cell obtainable from step c) and/or the mixture comprising induced pluripotent stem cells and non-pluripotent cells obtained in step a) is contacted with a Rho-associated kinase inhibitor for up to about 0.5, 1, 2, 4, 8, 12 hours, about 2, about 4, or about 6 days; or at least about 0.5, 1, 2, 4, 8, hours, about 2, about 4, or about 6 days; or any range derivable therefrom.

In one embodiment, the carbohydrate-binding protein is ECA and the Rho-associated kinase inhibitor is pinacidil or Y-27632.

In one embodiment, the carbohydrate-binding protein is ECA and the Rho-associated kinase inhibitor is pinacidil. This embodiment has the added utility that ECA together with pinacidil promotes a highly efficient expansion of the induced pluripotent cell obtainable from step c) and/or the mixture comprising induced pluripotent stem cells and non-pluripotent cells obtained in step a) without any impairment of quality.

In one embodiment, the carbohydrate-binding protein is ECA and the Rho-associated kinase inhibitor is Y-27632. This embodiment has the added utility that ECA together with Y-27632 promotes a highly efficient expansion of induced pluripotent stem cells without any impairment of quality.

In one embodiment, the method comprises:

step a) of inducing non-pluripotent cells to produce a mixture comprising induced pluripotent stem cells and non-pluripotent cells and cultivating the mixture comprising induced pluripotent stem cells and non-pluripotent cells;

step b) of contacting the mixture comprising induced pluripotent stem cells and non-pluripotent cells obtainable from step a) with the antibody K21;

step c) of selecting an induced pluripotent stem cell bound by the antibody K21; and

step d) of cultivating the induced pluripotent stem cell obtainable from step c),

wherein the induced pluripotent stem cell obtainable from step c) and/or the mixture comprising induced pluripotent stem cells and non-pluripotent cells obtained in step a) is contacted with a carbohydrate-binding protein and a Rho-associated kinase inhibitor simultaneously at one or more time intervals during the cultivation in step d) and/or in step a), and

wherein the carbohydrate-binding protein is ECA and the Rho-associated kinase inhibitor is pinacidil or Y-27632.

In one embodiment, the induced pluripotent stem cell obtainable from step c) and/or the mixture comprising induced pluripotent stem cells and non-pluripotent cells obtained in step a) is contacted with the carbohydrate-binding protein and the Rho-associated kinase inhibitor simultaneously when the induced pluripotent stem cell obtainable from step c) and/or the mixture comprising induced pluripotent stem cells and non-pluripotent cells obtained in step a) is seeded or passaged to the cultivation in step d) and/or in step a). In other words, the induced pluripotent stem cell obtainable from step c) and/or the mixture comprising induced pluripotent stem cells and non-pluripotent cells obtained in step a) is contacted with the carbohydrate-binding protein and the Rho-associated kinase inhibitor at a time interval starting from the starting point of the cultivation in step d). The end point of the time interval may be soon thereafter, e.g. when the induced pluripotent stem cell obtainable from step c) and/or the mixture comprising induced pluripotent stem cells and non-pluripotent cells obtained in step a) has effectively adhered. Alternatively, the end point of the time interval may be e.g. at the beginning of, during or after the expansion phase. This embodiment has the added utility that contacting the induced pluripotent stem cell obtainable from step c) and/or the mixture comprising induced pluripotent stem cells and non-pluripotent cells obtained in step a) with the carbohydrate-binding protein and the Rho-associated kinase inhibitor increases and improves the survival and adherence of the induced pluripotent stem cell obtainable from step c) and/or the mixture comprising induced pluripotent stem cells and non-pluripotent cells obtained in step a).

After seeding or passaging, the growth of cell cultures typically proceeds from the initial lag phase to the expansion phase. During the expansion or expansion phase, induced pluripotent stem cells proliferate and form an induced pluripotent stem cell population that expands spatially. Frequently, induced pluripotent cells proliferate exponentially during expansion; this growth is often also called the log phase. When induced pluripotent stem cells occupy all available substrate or matrix and have no room left for expansion, or when the culture medium no longer has the capacity to support further growth, induced pluripotent stem cell proliferation is greatly reduced or ceases, and the induced pluripotent stem cells enter the stationary phase.

In one embodiment, an induced pluripotent stem cell population derived from the induced pluripotent stem cell obtainable from step c) is contacted with the carbohydrate-binding protein and the Rho-associated kinase inhibitor simultaneously during the expansion of the induced pluripotent stem cell population.

In one embodiment, an induced pluripotent stem cell population derived from the induced pluripotent stem cell obtainable from step c) is contacted with the carbohydrate-binding protein, a matrix such as Matrigel or laminin, and/or the Rho-associated kinase inhibitor simultaneously during the expansion of the induced pluripotent stem cell population. In one embodiment, an induced pluripotent stem cell population derived from the induced pluripotent stem cell obtainable from step c) is contacted with a matrix such as Matrigel or laminin, and/or the Rho-associated kinase inhibitor simultaneously during the expansion of the induced pluripotent stem cell population.

In this context, the term “during the expansion” should be understood as referring to one or more time intervals during which the induced pluripotent stem cell population derived from the induced pluripotent stem cell obtainable from step c) and/or the mixture comprising induced pluripotent stem cells and non-pluripotent cells obtained in step a) proliferates and expands spatially.

In one embodiment, the induced pluripotent stem cell obtainable from step c) and/or the mixture comprising induced pluripotent stem cells and non-pluripotent cells obtained in step a) is contacted with the carbohydrate-binding protein and the Rho-associated kinase inhibitor simultaneously essentially during the entire duration of the cultivation in step d) and/or in step a).

In this context, the term “during the entire duration of the cultivation” should be understood as referring to all or essentially all time points and time intervals between the starting point of the cultivation and the end of the cultivation in step d) and/or in step a).

The carbohydrate-binding protein and the Rho-associated kinase inhibitor need not be contacted with the induced pluripotent stem cell obtainable from step c) and/or the mixture comprising induced pluripotent stem cells and non-pluripotent cells obtained in step a) simultaneously at all time intervals at which the carbohydrate-binding protein or the Rho-associated kinase inhibitor is contacted with the induced pluripotent stem cell obtainable from step c) and/or the mixture comprising induced pluripotent stem cells and non-pluripotent cells obtained in step a). In other words, the induced pluripotent stem cell obtainable from step c) and/or the mixture comprising induced pluripotent stem cells and non-pluripotent cells obtained in step a) may be contacted with the carbohydrate-binding protein and the Rho-associated kinase inhibitor partially at different time intervals, provided that the induced pluripotent stem cell obtainable from step c) and/or the mixture comprising induced pluripotent stem cells and non-pluripotent cells obtained in step a) is contacted with the carbohydrate-binding protein and a Rho-associated kinase inhibitor simultaneously at least at one time interval during the cultivation in step d) and/or in step a). For instance, the induced pluripotent stem cell obtainable from step c) and/or the mixture comprising induced pluripotent stem cells and non-pluripotent cells obtained in step a) may be contacted with the carbohydrate-binding protein during the entire duration of the cultivation in step d) and/or in step a), while the Rho-associated kinase inhibitor is contacted with the induced pluripotent stem cell obtainable from step c) and/or the mixture comprising induced pluripotent stem cells and non-pluripotent cells obtained in step a) only at one or more time intervals during the cultivation in step d) and/or in step a).

In one embodiment, the induced pluripotent stem cell obtainable from step c) and/or the mixture comprising induced pluripotent stem cells and non-pluripotent cells obtained in step a) is contacted with the carbohydrate-binding protein essentially during the entire duration of the cultivation in step d) and/or in step a) and the Rho-associated kinase inhibitor when the induced pluripotent stem cell obtainable from step c) and/or the mixture comprising induced pluripotent stem cells and non-pluripotent cells obtained in step a) is seeded or passaged to the cultivation in step d) and/or in step a).

In one embodiment, an induced pluripotent stem cell population derived from the induced pluripotent stem cell obtainable from step c) is contacted with the carbohydrate-binding protein essentially during the entire duration of the cultivation and with the Rho-associated kinase inhibitor during the expansion of the induced pluripotent stem cell population.

In one embodiment, the induced pluripotent stem cell obtainable from step c) and/or the mixture comprising induced pluripotent stem cells and non-pluripotent cells obtained in step a) is contacted with the carbohydrate-binding protein essentially during the entire duration of the cultivation in step d) and/or in step a) and with the Rho-associated kinase inhibitor when the induced pluripotent stem cell obtainable from step c) and/or the mixture comprising induced pluripotent stem cells and non-pluripotent cells obtained in step a) is seeded or passaged to the cultivation in step d) and/or in step a), and during the expansion of the induced pluripotent stem cell population derived from the induced pluripotent stem cell obtainable from step c) and/or the mixture comprising induced pluripotent stem cells and non-pluripotent cells obtained in step a) the amount of the Rho-associated kinase inhibitor is decreased.

In other embodiments, the induced pluripotent stem cell obtainable from step c) is contacted with a Rho-associated kinase inhibitor for at least one to five passages. Optionally, the Rho-associated kinase inhibitor is subsequently withdrawn from the culture medium, for example after about 0.5, 1, 2, 4, 8, 12 hours or after about 2, about 4, or about 6 days, or any range derivable therein. In other embodiments, the Rho-associated kinase inhibitor is withdrawn after at least one, two, three, four, five passages or more, or any range derivable therein.

In other embodiments, the concentration of the Rho-associated kinase inhibitor is reduced after at least one, two, three, four, five passages or more, or any range derivable therein, and the reduction is at least 2, 5, 10, 100, 1 000, 10 000, 100 000 or 1 000 000 fold compared to the original concentration.

In one embodiment, the concentration of the Rho-associated kinase inhibitor is reduced after one passage. In one embodiment, the reduction is at least 2, 5, 10, 100, 1 000, 10 000, 100 000 or 1 000 000 fold compared to the original concentration. In some embodiments, the concentration of the Rho-associated kinase inhibitor is reduced after about 0.1, 0.2, 0.5, 1, 2, 4, 8, 12 hours or after about 1, about 2, or about 4 days, or any range derivable therefrom. This embodiment has the added utility that the inhibitory activity to cell growth and proliferation is substantially reduced or essentially removed.

A culture vessel used for culturing the stem cell(s) can include, but is particularly not limited to: flask, flask for tissue culture, dish, petri dish, dish for tissue culture, multi dish, micro plate, micro-well plate, multi plate, multiwell plate, micro slide, chamber slide, tube, tray, culture bag, and roller bottle, as long as it is capable of culturing the induced pluripotent stem cell therein. Exemplary multiwell plates include, but are not limited to, a 6 well plate, a 12 well plate, a 24 well plate, a 48 well plate, a 96 well plate, and a 384 well plate. The stem cells may be cultured in a volume of at least or about 0.1, 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, or any range derivable therein, depending on the needs of the culture. In a certain embodiment, the culture vessel may be a bioreactor, which may refer to any device or system that supports a biologically active environment. The bioreactor may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with the carbohydrate binding protein. For differentiation purposes, the substrate for differentiated cell adhesion can be any material intended to attach cells. The substrate for cell adhesion may include the carbohydrate binding protein, collagen, gelatin, poly-L-lysine, poly-D-lysine, laminin, vitronectin, collagen and fibronectin and mixtures thereof, for example Matrigel or modified Matrigel such as Geltrex®.

An induced pluripotent stem cell obtainable from step c) or from step d) may be characterised in order to verify that the induced pluripotent stem cell obtained is fully reprogrammed. A common process of generating new induced pluripotent stem cell lines includes a step of identifying the formed induced pluripotent stem cell by their colony morphology and collecting the formed colonies as starting material for cell line generation (Takahashi, K., and Yamanaka, S., Cell 126:663-76, 2006). An induced pluripotent stem cell obtainable from step c) or from step d) may also be characterised by antibiotic selection or selection based on reporter gene expression or surface markers.

When the induced pluripotent stem cell culture has been maintained for the desired duration, the cultivation ends. Optionally, the induced pluripotent stem cells may be collected, for instance by detaching from the matrix or substrate by enzymatic and/or mechanical means. Collected induced pluripotent stem cells may be seeded or passaged further, or they may be differentiated to a differentiated cell type or a differentiated lineage, or either the induced pluripotent stem cells or the differentiated cells may be used e.g. as a medicament or for other clinical applications. Exemplary uses for the induced pluripotent stem cells or the differentiated cells are reviewed in e.g. D. A. Robinton and G. Q. Daley, Nature 481:295-305, 2012.

The present invention also relates to an induced pluripotent stem cell or an induced pluripotent stem cell population obtainable by the method according to one or more embodiments of the invention.

In one embodiment, the induced pluripotent stem cell population is monoclonal.

In one embodiment, the induced pluripotent stem cell or induced pluripotent stem cell population is free of feeder cells and/or xeno-free.

The present invention also relates to a composition comprising the induced pluripotent stem cell or the induced pluripotent stem cell population obtainable by the method according to one or more embodiments of the invention.

The present invention also relates to a composition comprising the induced pluripotent stem cell or the induced pluripotent stem cell population obtainable by the method according to one or more embodiments of the invention for use as a medicament.

The present invention also relates to a kit for generating induced pluripotent stem cells, wherein the kit comprises a binding agent and a carbohydrate-binding protein,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal, and

wherein the carbohydrate-binding protein is capable of binding the non-reducing terminal oligosaccharide structure according to the formula

(Fucα1-2)ₙGalβ1-4GlcNAc,

wherein n=0 or 1.

In the context of the kit, the binding agent may be any embodiment of the binding agent described in this document.

In the context of the kit, the carbohydrate-binding protein may be any embodiment of the carbohydrate-binding protein described in this document.

In one embodiment, the kit further comprises a Rho-associated kinase inhibitor. The Rho-associated kinase inhibitor may be any Rho-associated kinase inhibitor described in this document.

The kit may optionally comprise a further component, such as instructions for using the kit.

The present invention further relates to a culture system for generating induced pluripotent stem cells, wherein the culture system comprises a culture vessel, a culture medium, a binding agent and optionally a carbohydrate-binding protein,

wherein the binding agent is capable of binding an epitope consisting of the non-reducing terminal saccharide structure according to formula

(Fucα1-2)ₐGalβ1-3HexNAcβ,

wherein a=0 or 1 and Hex is either Glc or Gal, and

wherein the carbohydrate-binding protein is capable of binding the non-reducing terminal oligosaccharide structure according to the formula

(Fucα1-2)ₙGalβ1-4GlcNAc,

wherein n=0 or 1.

In the context of the culture system, the binding agent may be any embodiment of the binding agent described in this document.

In one embodiment, the binding agent is K21, anti-PLN or xeno-free anti-PLN.

In the context of the culture system, the carbohydrate-binding protein may be any embodiment of the carbohydrate-binding protein described in this document.

In one embodiment, the culture system further comprises a Rho-associated kinase inhibitor. The Rho-associated kinase inhibitor may be any Rho-associated kinase inhibitor described in this document.

The culture vessel may be any culture vessel described in this document.

In one embodiment, the culture system further comprises a matrix. The matrix may be any matrix described in this document.

In one embodiment, the culture medium is a defined medium and/or a xeno-free medium.

In one embodiment, the culture system is essentially free of feeder cells.

In one embodiment, the culture medium and/or matrix do not comprise inhibitory amounts of inhibitory non-reducing terminal oligosaccharide structures according to the formula

(Neu5R¹α2-3)ₘ(Fucα1-2)ₙGalβR²,

wherein R¹ is Ac or Gc;

m=0 if the carbohydrate-binding protein is not a galectin;

m=0 or 1 if the carbohydrate-binding protein is a galectin;

n=0 or 1-m; and

R² is 1-4GlcNAc or 1-4Glc or absent.

In this context, the term “inhibitory amounts” should be understood as referring to amounts that do not significantly inhibit adherence of the induced pluripotent stem cell obtainable from step c). Glycoproteins used in cell culture media and matrices such as transferrin, serum proteins, and extracellular matrix components such as fibronectin and laminin may comprise the inhibitory non-reducing terminal oligosaccharide structures depending on the source and manufacturing process of the glycoprotein.

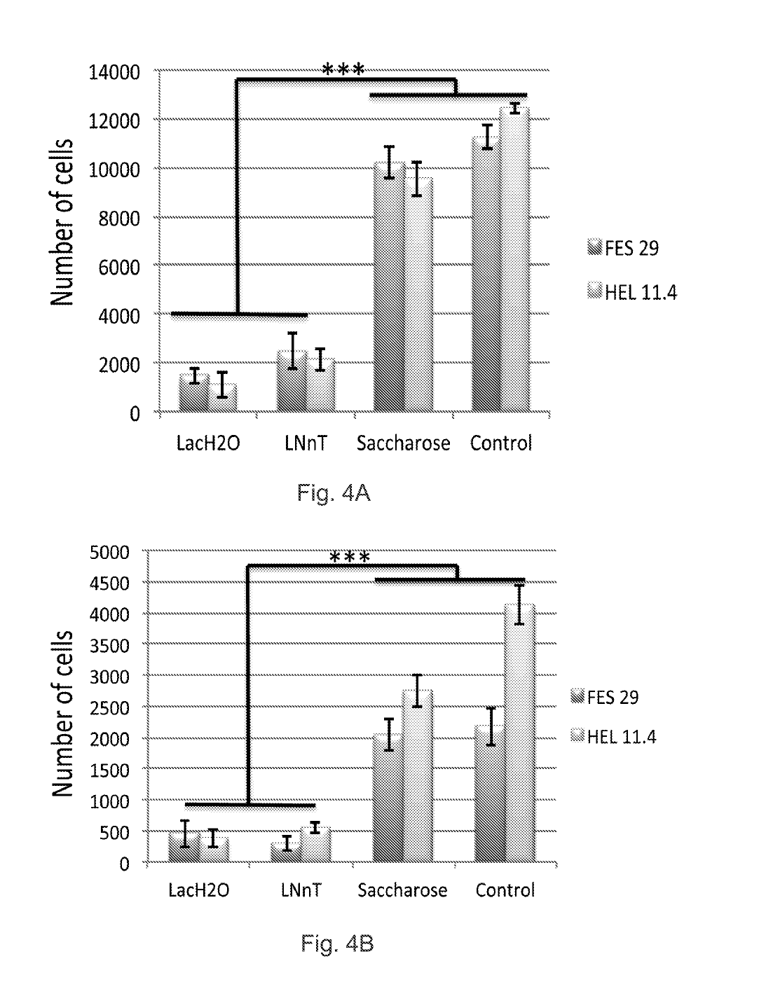

The amounts of the inhibitory non-reducing terminal oligosaccharide structures that may be inhibitory may be determined by inhibition experiments (described in e.g. Example 3 and FIG. 4).

In one embodiment, the amount of free oligosaccharide containing the inhibitory non-reducing terminal oligosaccharide structures is less than 100 mM. This embodiment has the added utility that the inhibition of adherence of induced pluripotent stem cells is significantly reduced.

In one embodiment, the culture medium comprises transferrin.

Transferrin is often added to stem cell culture media and it is an essential component of validated induced pluripotent stem cell culture systems. Transferrin that is available in industrial scale is often isolated from animal serum, most often bovine serum, or from human serum. Examples of such products used as stem cell medium supplements were analysed in Example 4 and found to contain variable amounts of the inhibitory non-reducing terminal oligosaccharide structures. In serum transferrins, the specific amount of the inhibitory non-reducing terminal oligosaccharide structures was found to depend on the level of sialylation and vary from product to product. Since the product specifications of cell culture media often do not disclose amounts of specific glycoprotein components or amounts of the inhibitory non-reducing terminal oligosaccharide structures, it is impossible to know without analysing or testing a medium whether it will inhibit the method of the present invention. Since amounts of the inhibitory non-reducing terminal oligosaccharide structures may also vary significantly from lot to lot, e.g. due to variable source of material, animal age or condition, or manufacturing process, it is also impossible to know by testing one lot of a supplement whether a next lot will be similar with regard to amount of the inhibitory non-reducing terminal oligosaccharide structures.

In one embodiment, the amounts of the inhibitory non-reducing terminal oligosaccharide structures in the form of non-sialylated serum transferrin N-linked glycan antennae are within the range of 1.5 nM to 5 μM. In one embodiment, the amount of the inhibitory non-reducing terminal oligosaccharide structures depends on the source and glycoform of the supplement and on the formulation of the medium.

In one embodiment, the culture medium and matrix comprise less than 100 mM, 10 mM, 1 mM, 100 μM, 10 μM, 5 μM, 1 μM, or 0.3 μM of the inhibitory non-reducing terminal oligosaccharide structures. In an embodiment, the culture medium comprising transferrin comprises between 1.5 nM and 50 μM, between 5 nM and 5 μM, between 5 nM and 1 μM, or between 25 nM and 0.3 μM of the inhibitory non-reducing terminal oligosaccharide structures.

In one embodiment, the culture medium and matrix does not comprise the inhibitory non-reducing terminal oligosaccharide structures.

In one embodiment, the culture system contains only non-glycoprotein components such as recombinant proteins produced in bacteria. In one embodiment, the carbohydrate-binding protein is a non-glycoprotein. In one embodiment, the carbohydrate-binding protein is non-glycosylated ECA such as recombinantly produced ECA. A recombinantly produced ECA is described e.g. by Stancombe et al. (2003, Protein Expr. Purif. 30:283-92).

In an embodiment, the culture system contains selected or produced glycoprotein components with controlled glycosylation with regard to the inhibitory non-reducing terminal oligosaccharide structures.

In one embodiment, the culture system comprises culture medium comprising transferrin. In some embodiments, the culture medium comprises transferrin in concentrations between 0.5 mg/l and 1 mg/l, between 0.5 mg/l and 100 mg/l, between 1 mg/l and 50 mg/l, or between 5 mg/l and 20 mg/l. In an embodiment the transferrin is serum transferrin. In an embodiment the transferrin is human serum transferrin. In an embodiment the transferrin is sialylated with nLN between 0 (fully sialylated) and 0.5 (50% sialylated) as defined for nLN in Example 4. In some embodiments, the transferrin is sialylated with nLN between 0.1 and 0.5, between 0.2 and 0.4 or between 0.2 and 0.35. In one embodiment, the transferrin is sialylated with Neu5Ac. In some embodiments, the Neu5Gc content of the transferrin is below 5%, below 1%, below 0.1%, below 0.01% or below 0.001% of the total amount of Neu5Ac and Neu5Gc.

In one embodiment, the culture medium has pH controlled between 6.8 and 7.8.

In one embodiment, the culture system comprises cell culture medium with controlled pH. In this embodiment, the formation of the inhibitory non-reducing terminal oligosaccharide structures from sialylated glycoproteins, such as transferrin, may be avoided. Human lysosomal sialidase and cytosolic sialidase have pH optima at about 4.5 and 6.5, respectively. In case medium pH is lowered to from about 6.5 to about 7.0, their release into the culture medium during stem cell culture can generate the inhibitory non-reducing terminal oligosaccharide structures from sialylated glycoproteins such as transferrin. Further, sialylated glycoproteins such as transferrin are known to undergo acid-catalyzed desialylation to asialoglycoprotein comprising the inhibitory non-reducing terminal oligosaccharide structures in acidic pH. However, induced pluripotent stem cell cultures are often controlled only by phenol red colour indicator comprised in the medium. Phenol red changes colour to yellow in the pH range from about 6.5 to about 6.8. Thus the precise control of the medium pH may be highly advantageous when using sialylated glycoprotein supplements such as transferrin. In one embodiment, the medium pH is controlled to pH range not lower than from about 6.8 to about 7.0.

In an embodiment, the pH of the medium comprised in the culture system is controlled above the pH optima of sialidases or to or above neutral pH 7.0. In this embodiment, the generation of the inhibitory non-reducing terminal oligosaccharide structures may be avoided. pH ranges from 6.8 to 7.6 are generally acceptable for mammalian cell culture and from 6.6 to 7.8 are such that mammalian cells can survive in cell culture, while pH ranges from 7.2 to 7.4 are optimal for mammalian cell culture. Methods to control the medium pH in cell culture are known in the art and include e.g. choosing medium with higher buffer capacity, timely medium changes, pH measurement during culture, and changing the medium pH by addition of e.g. carbon dioxide to lower pH or carbonate salt to increase pH.

In one embodiment, the medium comprised in the culture system comprising sialylated glycoprotein such as transferrin has pH from 6.8 to 7.8, from 7.0 to 7.8, from 7.0 to 7.6, from 7.1 to 7.5, or from 7.2 to 7.4.

In one or more embodiments, the culture system is automated.

In one or more embodiments, the culture system comprises means for automating steps a-d, b-d, or c-d of the method according to one or more embodiments of the invention.

The culture system may further comprise an apparatus for handling, culturing and/or dispensing non-pluripotent cells, a mixture of cells comprising induced pluripotent cells and non-pluripotent cells and/or selected induced pluripotent stem cells. The apparatus may further be suitable for handling, culturing and/or dispensing a mixture of cells comprising induced pluripotent cells and non-pluripotent cells and/or selected induced pluripotent stem cells. The apparatus may further comprise means for handling, culturing and/or dispensing non-pluripotent cells, a mixture of cells comprising induced pluripotent cells and non-pluripotent cells and/or selected induced pluripotent stem cells. The apparatus may further comprise means for handling and/or dispensing culture medium. Such means may comprise e.g. a liquid handler robot and/or an incubator.

In one embodiment, the culture system further comprises an apparatus for handling, culturing and/or dispensing non-pluripotent cells, a mixture of cells comprising induced pluripotent cells and non-pluripotent cells and/or selected induced pluripotent stem cells, such as a liquid handler robot, a cell plating unit, and/or an incubator; and an apparatus for selecting induced pluripotent stem cells, such as a sorting unit, an apparatus for fluorescent-activated cell sorting (FACS), magnetic-activated cell sorting or affinity chromatography.

In one embodiment, the culture system further comprises a culture of non-pluripotent cells.

The culture system or apparatus may further comprise means for induction or reprogramming of non-pluripotent cells, such as an induction unit; and/or means for contacting the mixture comprising induced pluripotent stem cells and non-pluripotent cells with the binding agent.

The culture system or apparatus may also comprise means for selecting induced pluripotent stem cells bound by the binding agent. For example, the apparatus may comprise a FACS for selecting induced pluripotent stem cells bound by the binding agent. The culture system or apparatus may comprise means for changing medium, contacting induced pluripotent stem cells with means for mechanical or chemical separation of induced pluripotent stem cells, means for incubation, means for cell separation, means for passaging cells and/or means for differentiating induced pluripotent stem cells. Such means may comprise e.g. a liquid handler robot for induction of non-pluripotent cells, handling, dispensing and/or changing medium or non-pluripotent cell and iPSC passaging. A wide array of liquid handler robots are known in the art and may be used according to the invention. Furthermore, it is contemplated that an automated system or apparatus may comprise a bioreactor for mediating fluid transfer and/or cell seeding/passaging by pumps or pressure gradients.

The culture system or apparatus may also comprise means for selecting or sorting induced pluripotent stem cells into the culture vessel and/or means for culturing and expanding induced pluripotent stem cells, such as an expansion unit.

In one embodiment, the culture system or an apparatus for automated selection, maintenance and expansion of induced pluripotent stem cells comprises means for mechanical or chemical separation of induced pluripotent stem cells. Means for mechanical separation of induced pluripotent stem cells may include e.g. a cell scraper, means for trituration such as a narrow bore pipette, means for fine needle aspiration, means for vortex disaggregation such as a vortex mixer, or means for forced filtration through a fine nylon or stainless steel mesh such as a filter comprising a fine nylon or stainless steel mesh. Means for chemical separation may comprise e.g. chelating molecules (e.g., EDTA, EGTA, citrate or similar molecules) or a proteolytic enzyme. In one embodiment, the culture system comprises means for removing culture medium from the induced pluripotent stem cells and means for contacting the iPSCs with a proteolytic enzyme or chemical, and means for incubating the iPSCs to separate iPS cell clusters. Such a system may comprise means for incubating iPSCs with the proteolytic enzyme or chemical for between about 2 and about 10 minutes, or at most about 3, 4, 5, 6, 7, 8 or 9 minutes and means for subsequent removal of the protease or chemical. The system may further comprise means for seeding or passaging iPSCs into culture vessels, for example, in the frequency of one or 2-10 iPSC per culture vessel, such as a well of a multiwell plate. In one embodiment, the means for separating cells may comprise a combination of means for mechanical separation and means for enzymatic separation.

In one embodiment, a system or apparatus for automated selection and expansion of iPS cells comprises an incubator, a liquid handler unit, liquid containers comprising culture media, cell detachment reagents, buffer, and/or an operating program for cell selection, separation, and expansion.

In one embodiment, the culture system further comprises induced pluripotent stem cells comprising the non-reducing terminal saccharide structure according to formula

(Fucα1-2)ₐGalβ1-3HexNAcβ,

wherein a=0 or 1 and Hex is either Glc or Gal.

The culture system or apparatus may comprise one or more of the following apparatuses: a computer, a computer interface, liquid reservoirs or containers for medium and supplements, a pipetting unit, an air pressure or hydraulic unit, a cell detachment reagent container (for means for chemical separation), a reservoir for binding agent, a reservoir for secondary binding agent, one or more incubator units, a plate unit, means for moving plate units, such as a robotic arm or a conveyor belt, a power unit.

The culture system may also comprise an apparatus for implementing the principles of the present invention, the apparatus comprising at least one memory, and at least one processor coupled with the at least one memory; wherein the at least one memory comprises program code instructions, which when executed by the at least one processor, cause the apparatus to perform the steps described above in the context of the method aspect.

The at least one memory and the at least one processor may be implemented e.g. in the form of one or more computers, wherein suitable computer program code is installed for performing the method steps.

In one embodiment, the apparatus comprises at least one memory, and at least one processor coupled with the at least one memory; wherein the at least one memory comprises program code instructions, which when executed by the at least one processor, cause the apparatus to perform an automated method for serial expansion of iPSCs comprising (a) obtaining a first population of iPS cells in growth media, (b) separating the iPS cells with an automated separation system, (c) suspending the separated cells in fresh growth media to provide an expanded population of iPS cells, (d) incubating the expanded iPS cell population under conditions supporting cell growth and (e) repeating steps b-d one or more times to provide a serially expanded population of iPS cells. Thus, methods of the invention may be used for the passage or expansion of a population of induced pluripotent stem cells for any number of passages from initial iPSC culture to senescence or differentiation of the iPS cells.

For example, the at least one memory may comprise program code instructions, which when executed by the at least one processor, cause the apparatus to perform the steps of (i) contacting heterogeneous mixture of non-pluripotent cells and induced pluripotent stem cells with a binding agent such as K21 antibody, (ii) selecting and/or isolating K21+iPS cells into culture vessels such as 96 well plate or 384 well plate in frequency of one iPS cell per well or 2-10 iPS cells per well, (iii) cultivating the iPS cells in a culture medium. Further, the at least one memory may comprise program code instructions, which when executed by the at least one processor, cause the apparatus to perform the steps of contacting iPS cells with a proteolytic enzyme or chemical, (iii) incubating the iPS cells with a proteolytic enzyme or chemical to separate iPS cell clusters and/or (iv) subjecting the incubated iPS cells to mechanical agitation to further separate iPS cell clusters. In some embodiments, the at least one memory may comprise program code instructions, which when executed by the at least one processor, cause the apparatus to perform the steps of moving iPS cells and or fluids between different chambers in the system. In some embodiments, cell culture plates may be moved for one chamber to another (e.g., into or out of an incubator).

In some embodiments, the culture system may be suitable for screening one or more candidate compounds which may modulate the differentiation state of an iPS cell. For example, the candidate compound may promote differentiation of a stem cell towards a specific lineage (e.g., hematopoietic, etc.). In other embodiments, the candidate compound may promote de-differentiation or maintain a de-differentiated state in a cell (e.g., promote the generation of an iPS cell from a fibroblast or other cell).

Induced pluripotent stem cells generated according to one or more embodiments of the present invention express surface markers such as Tra-1-60 that are characteristic to pluripotent stem cells and are capable of forming induced pluripotent stem cell colonies and retaining these characteristics during cultivation.

One or more embodiments of the present invention significantly increase the efficiency of generation of induced pluripotent stem cells. A high proportion of induced pluripotent stem cells obtainable by one or more embodiments of the present method are fully reprogrammed induced pluripotent stem cells. Thus more induced pluripotent stem cells may be generated from a single induction. The induced pluripotent stem cells can be selected a short time period after induction, as there is no need to passage and/or screen colonies obtained from the induced cells. Thus stable, viable induced pluripotent stem cells (e.g. colonies or cell lines) free of non-pluripotent cells may be obtained within weeks of the induction, e.g. within 2 or 3 weeks of the induction in step a). Lengthy steps of culturing a mixture comprising induced pluripotent stem cells and non-pluripotent cells, selection and passaging of individual colonies—frequently for a number of rounds—may be partially or fully dispensed with. Thus the time, effort and costs required for generating an induced pluripotent cell line are reduced. Induced pluripotent stem cells may, according to one or more embodiments of the present invention, be selected at an early stage after induction, e.g. at a stage where the cells comprised in the mixture comprising induced pluripotent stem cells and non-pluripotent cells obtainable from step a) has not yet formed colonies. Furthermore, homogeneous and/or monoclonal induced pluripotent cell lines may be obtained.

As induced pluripotent stem cells are obtained with a very high frequency, only a small population of non-pluripotent cells needs to be induced in order to obtain a significant number of induced pluripotent stem cells. This reduces costs significantly.

A very high proportion, even up to 100%, of cells obtainable by one or more embodiments of the method, are stable induced pluripotent stem cells. The method is thus very selective and efficient.

Further, the method is amenable for automation.

The embodiments of the invention described hereinbefore may be used in any combination with each other. Several of the embodiments may be combined together to form a further embodiment of the invention. A method, or a product to which the invention is related, may comprise at least one of the embodiments of the invention described hereinbefore.

### EXAMPLES

In the following, the present invention will be described in more detail. Reference will now be made in detail to the embodiments of the present invention, examples of which are illustrated in the accompanying drawings. The description below discloses some embodiments of the invention in such detail that a person skilled in the art is able to utilize the invention based on the disclosure. Not all steps of the embodiments are discussed in detail, as many of the steps will be obvious for the person skilled in the art based on this specification.

### Example 1

### Induction and Sorting of Cells

Human foreskin fibroblast (hFF) cells (CRL-2429; ATCC, Mananas, Va., USA) were infected by using retrovirus-induced over-expression of Oct4, Sox2, NANOG and LIN28 as described (Hussein, S. M., et al., 2011, Nature 471(7336):58-62). Three days after infection the cells were frozen.

Frozen infected cells were thawed and cultured for either 7, 10 or 12 additional days on Matrigel (plated 7800 cells/cm²) in HES medium: Knockout Dulbecco's Modified Eagle's Medium or DMEM/F12 supplemented with 20% Knockout SR, 2 mM Glutamax, 1% non-essential amino acids (all from Gibco, Life Technologies), 0.1 mM 2-mercaptoethanol (Sigma-Aldrich), bFGF (Biosource/Life Technologies) at 6 ng/ml, 0.25 mM sodium butyrate (Sigma-Aldrich) and 2 μM SB 431542 (Sigma-Aldrich). Control iPS cell line H9 (Hussein et al. 2011) was grown on Matrigel for the experiments.

At day 10, day 13 and day 15 after infection the cells were incubated with fluorescently labeled Tra-1-60 (Millipore) and K21 (Abcam) antibodies. By fluorescence-activated cell sorting (FACS) the single-positive, the double-positive, and the double negative populations were sorted onto 24-well plates (2000 cells/well) either onto ECA lectin in murine embryonic fibroblast (MEF) conditioned HES medium or onto Matrigel in E8 medium (Life technologies) to evaluate the ability of each cell population to form iPS cell colonies.

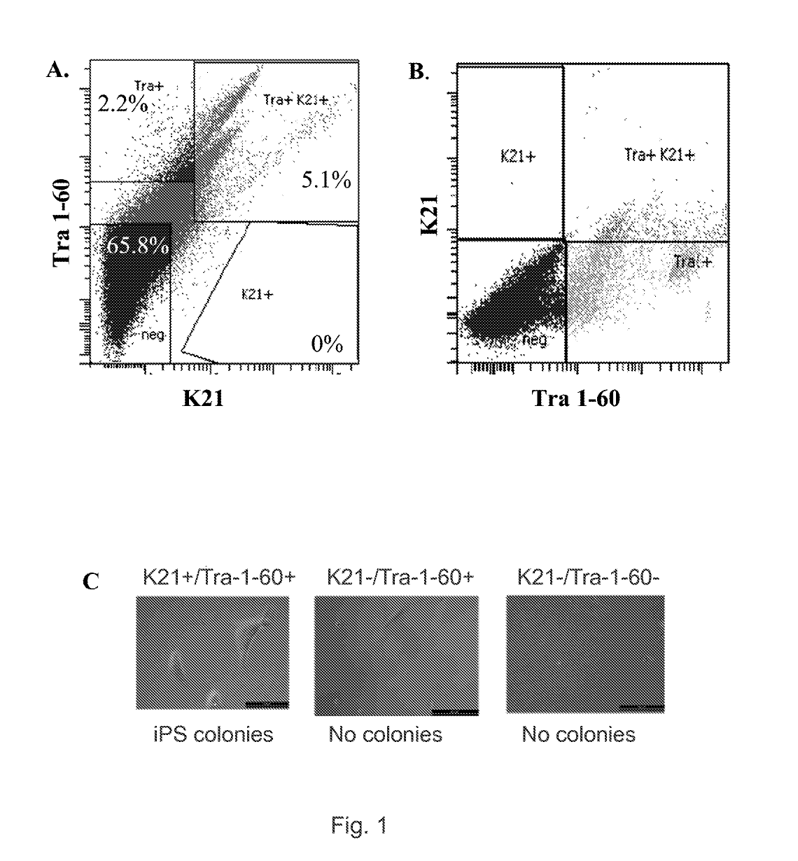

FIG. 1 shows the results of the experiment at day 10 and at day 15. At day 15 after infection, all K21 positive cells were also Tra-1-60 positive (5.1% K21+/Tra-1-60+ cells; FIG. 1A) and were able to form iPS cell colonies when plated on either ECA matrix in MEF conditioned medium (FIG. 1C) or Matrigel in E8 medium. However, K21 negative cells including both K21−/Tra-1-60+ and K21−/Tra-1-60− cells were not able to form colonies (FIG. 1C). At day 13, all K21 positive cells (both K21+/Tra-1-60+ cells and K21+/Tra-1-60− cells) were similarly able to form iPS cell colonies. FIG. 1B shows that at day 10 after infection a smaller proportion of the cells were K21 positive. No colonies were observed when either K21+/Tra-1-60+, K21−/Tra-1-60+ or K21−/Tra-1-60− cells were sorted and plated at day 10, showing that viable iPS cell colonies could be formed after day 10 after infection in the present conditions.

### Example 2

### Coating of Plates with ECA

ECA lectin (Sigma-Aldrich) solution (1 mg/ml in PBS) was let to passively adsorb onto surface of the cell culture plates (5 μg/cm²) (Nunc, Roskilde, Denmark or Costar, Corning Life Sciences, MA, USA) o/n at +4° C. followed by washing twice with PBS. The coated plates were stored at +4° C. and used within four weeks.

### Example 3

### Testing of Lectins

Three hESC lines (FES 29, FES 30 and H9) and two induced pluripotent stem (hiPS) cell line (FiPS 5-7 and HEL11.4) were included and cultured on Matrigel as previously described (Vuoristo S, Virtanen I, Takkunen M, Palgi J, Kikkawa Y, et al. (2009) Laminin isoforms in human embryonic stem cells: synthesis, receptor usage and growth support. J Cell Mol Med 13: 2622-2633; Mikkola M, Olsson C, Palgi J, Ustinov J, Palomaki T, et al. (2006) Distinct differentiation characteristics of individual human embryonic stem cell lines. BMC Dev Biol 6: 40). HEL11.4 was generated from adult fibroblasts (male, 84 years old) using retrovirus-induced overexpression of four genes: Oct-4, Sox2, Klf4, and c-Myc. Cells were infected with equal parts of hES medium and virus-containing supernatant twice at 24-h intervals. Cells were harvested and re-seeded on mitotically inactivated treated mouse embryonic fibroblast (mEF) layer three days after infection. Twenty-four days post-transduction, ES-like colonies were picked, expanded, and characterized.

Cells were passaged by using 0.1 mg/ml collagenase IV (Invitrogen) for 5 min at +37° C. and harvested onto ECA (Sigma-Aldrich) and Matrigel (Becton Dickinson) coated plates and cultured either in StemPro® or in mEF conditioned-medium (CM) (KO-DMEM supplemented with 20% KO-SR, 2 mM Glutamax, 0.1 mM β-mercaptoethanol, 0.1 mM non-essential amino acids (NEAA), all from Invitrogen, Carlsbad, Calif., USA) and supplemented with 8 ng/ml recombinant human bFGF (Invitrogen, Carlsbad, Calif., USA). Pinacidil (Sigma-Aldrich, 100 μM) was added to culture media during passaging. In some experiments, Y-27632 (10 μM) was added to culture media during passaging. In all experiments Matrigel™ (BD Biosciences) was used as control matrix. The Matrigel plates were prepared as recommended by the manufacturer.

ECA (Erythrina cristagalli agglutinin, binding specificity in type 2 N-acetyllactosamine structures), MAA (Maackia amurensis agglutinin, specific for α2,3-linked sialic acid), WFA (Wisteria floribunda agglutinin, binding preferentially to N-acetylgalactosamine in α- or β-linkage) and PWA (Phytolacca americana agglutinin, with N-acetylglucosamine specificity, binding also to polylactosamine structures) were tested for their ability to act as a growth supporting matrix for ESC lines FES 29 and FES 30 in mEF-CM medium. In cell culture conditions, stem cells attached onto ECA and MAA. Furthermore, continuous growth was acquired on ECA matrix.

### Flow Cytometry Analysis of Surface Antigens

Single cell suspensions were generated by incubation with TrypLE (Gibco) for 5 min at +37° C. Cells were stained with specific cell surface antibodies (SSEA-1, SSEA-3, Tra 1-60, H type 1, CXCR4) and fluorescein conjugated secondary antibody prior to analysis by flow cytometry (FACS Calibur, BD Biosciences). Antibodies are listed in Table 1.

TABLE 1
Antibodies used for analysis.

| | Manufacturer/catalog |
|---|---|
| Primary antibody | number |
| SSEA-3 | Millipore/MAB 4303 |
| Tra 1-60 | Millipore/MAB4360 |
| H-type1 | Abcam/Ab3355 |
| SSEA- 1 | Millipore/MAB4301 |
| CXCR4 | BD/555974 |
| Oct-4 | Santa Cruz |
| | Biotechnology/sc9081 |
| Nanog | Cell Signalling/4903 |
| Sox2 | Cell Signalling/3579 |
| Anti-Human Alpha-1- | DAKO/A0008 |
| Fetoprotein | |
| FOXA2 | Santa Cruz |
| | Biotechnology/sc-9187 |
| Anti-Human/Mouse | R&D Systems |
| Serum Albumin | |

### Immunohistochemistry of the Cells on ECA

Cells were fixed with 4% paraformaldehyde and permeabilized with 0.1% Triton X-100 (Sigma-Aldrich, St. Louis, Mo., USA) if needed. The antibodies used are listed in Table 1. The cells were probed with secondary antibodies for 30 min in the dark at RT. Cells were mounted using Vectashield mounting media with 4,6-diamidino-2-phenylindole (DAPI, Vector Laboratories, Burlingame, Calif.).

### Teratoma Formation

Cells were harvested with collagenase IV from ECA and Matrigel plates, and ca. 100,000 cells from each matrix were injected into nude mouse testis. After 7-8 weeks, tumors were dissected, fixed with 4% PFA and H&E stained sections histologically examined. The animal experiments were approved by the experimental animal welfare committee of the District Government of Southern Finland.

### RNA Isolation and Quantitative PCR

Total RNA was isolated using NucleoSpinR RNA II (Macheray-Nagel GmbH & Co. KG, Germany) according to manufacturer's instruction. Complementary DNA was synthesized from 50 μg of total RNA using iScript™ cDNASynthesis Kit (Biohit) according to manufacturer's instructions.

Real time SYBR Green quantitative PCR (qPCR) analyses was performed with Corbett Rotor-Gene 6000 (Corbett Life Science, Sydney, Australia) using the following conditions: 95° C. 7 minutes and 40 cycles of 95° C., 20 s; 56° C., 20 s; 72° C., 20 s. The data was analyzed according to comparative Ct method (Applied Biosystems, User Bulletin #2). Cyclophilin gene expression was an internal reference for normalization. All samples and controls were analyzed in duplicates. Primers used for qPCR are shown in Table 2.

TABLE 2
Primers used in qPCR analysis.

| Primer | 5′- | 3′- |
|---|---|---|
| Oct4 | TTGGGCTCGAGAAGG | TCCTCTCGTTGTGCA |
| | ATGTG | TAGTCG |
| | (SEQ ID NO: 21) | (SEQ ID NO: 22) |
| Sox2 | GCCCTGCAGTACAAC | TGCCCTGCTGCGAGT |
| | TCCAT | AGGA |
| | (SEQ ID NO: 23) | (SEQ ID NO: 24) |
| Nanog | CTCAGCCTCCAGCAG | TAGATTTCATTCTCT |
| | ATGC | GGTTCTGG |
| | (SEQ ID NO: 25) | (SEQ ID NO: 26) |
| Brachuyru | GCATGATCACCAGCC | TTAAGAGCTGTGATC |
| | ACTG | TCCTC |
| | (SEQ ID NO: 27) | (SEQ ID NO: 28) |
| Goosecoid | GAGAACCTCTTCCAG | TTCTTAAACCAGACC |
| | GAGAC | TCCAC |
| | (SEQ ID NO: 29) | (SEQ ID NO: 30) |
| Cyclophilin | CAATGGCCAACAGAG | CCAAAAACAACATGA |
| | GGAAG | TGCCA |
| | (SEQ ID NO: 31) | (SEQ ID NO: 32) |
| FOXA2 | AAGACCTACAGGCGC | CGTTGAAGGAGAGCG |
| | AGCT | AGTG |
| | (SEQ ID NO: 33) | (SEQ ID NO: 34) |
| CER1 | CATTGGGAGACCTGC | CCCAAAGCAAAGGTT |
| | AGGAC | GTTCTG |
| | (SEQ ID NO: 35) | (SEQ ID NO: 36) |

### PCR Arrays

FES 29 cells were cultured for 9 passages on ECA or Matrigel in CM media. Total RNA was isolated from three separate plates using RNeasy Mini kit (Qiagen, Valencia, Calif.) and complementary DNA was synthesized from 1 μg of total RNA using RT² First Strand Kit and RT² qPCR Master Mixes (SABiosciences) according to manufacturer's instruction. The RT² qPCR primer Assays (SABiosciences) were used to study the gene expression profile of genes related to the identification, growth and differentiation of stem cells (array PAHS-081).

### Karyotype Analysis

Karyotype was detected by G-banding technique in cytogenetics laboratory of the Yhtyneet Medix Laboratories Inc, Helsinki, Finland. Twenty metaphases were examined from each sample.

Basic Characteristics of hPSCs Cultured on ECA

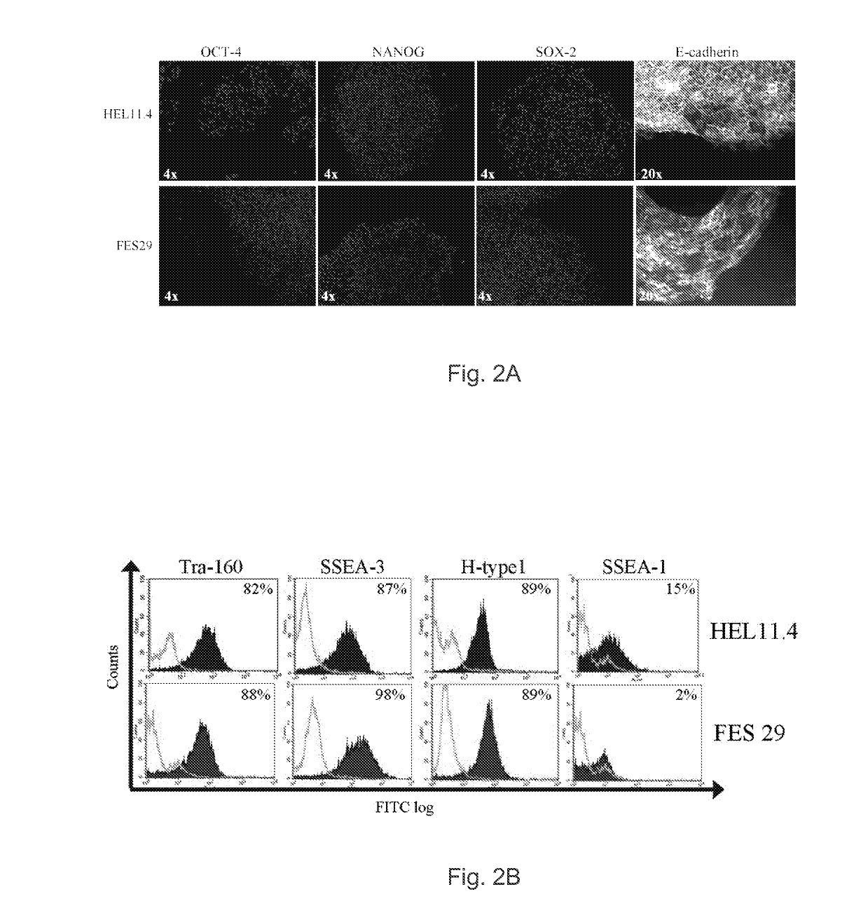

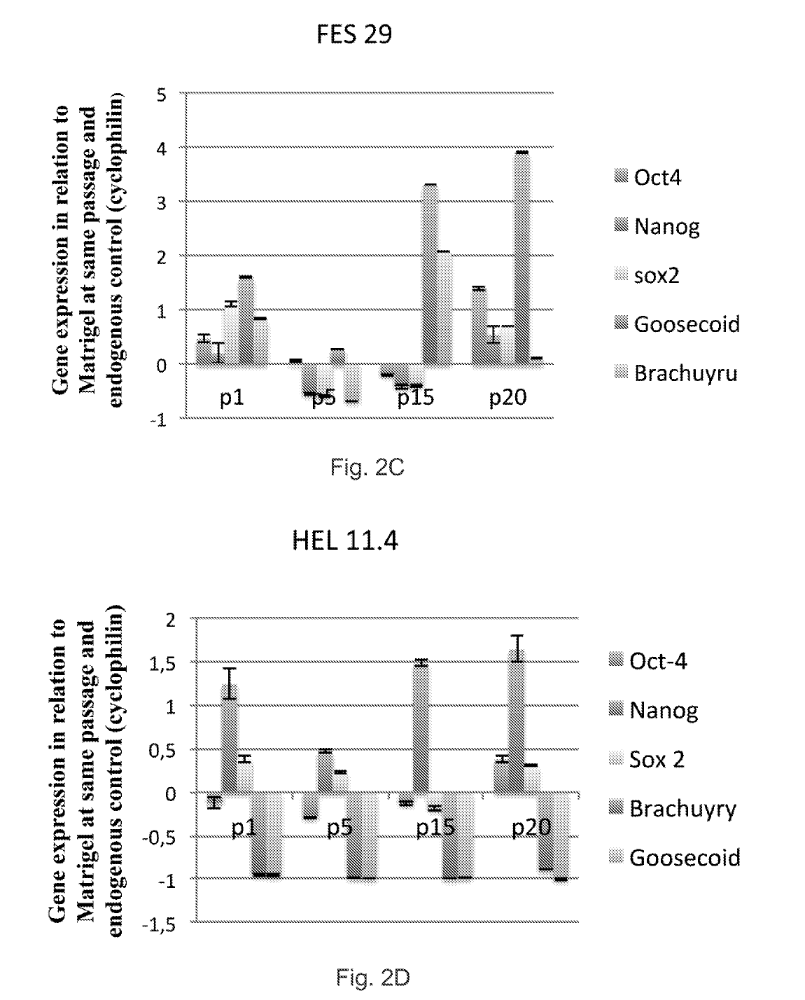

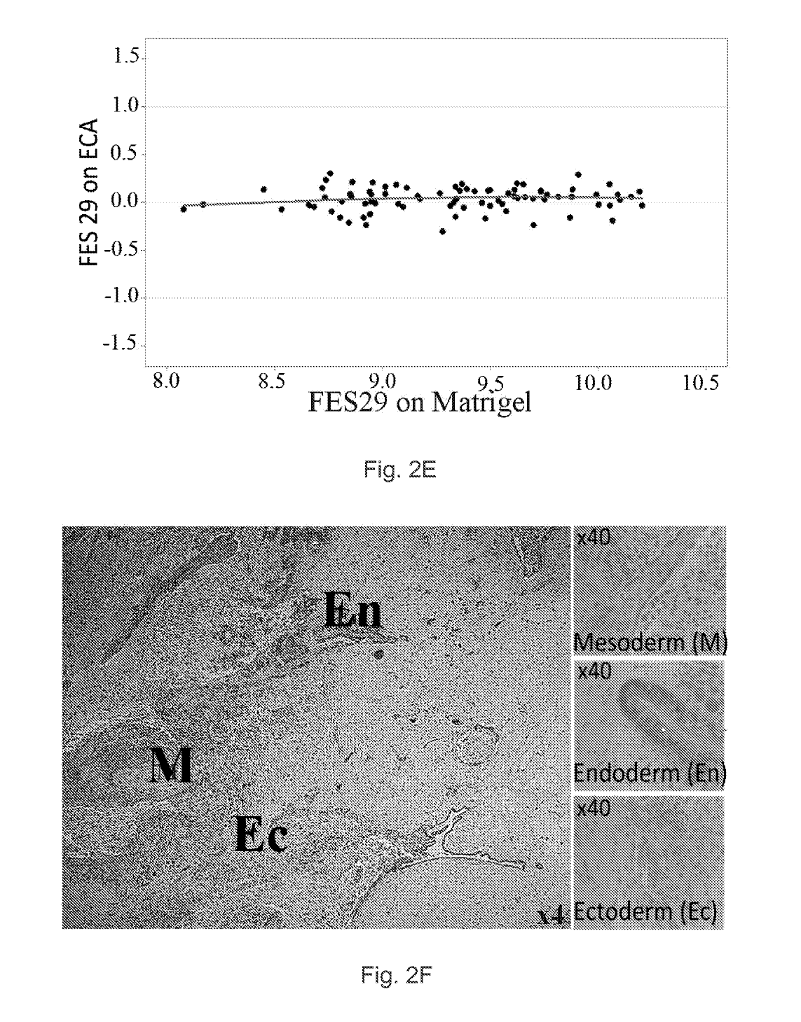

Long-term culturing on ECA-coated plates was evaluated with hPSCs lines (FES29 and HEL 11.4) and the results were compared to the same cell lines cultured on Matrigel. For most of the experiments the cells were cultured in mEF conditioned media and treated with the Rho-associated kinase inhibitor pinacidil during passaging. Without pinacidil the cells did not attach as effectively and they also partly changed morphology forming a lot of feeder-like cells. In long-term cultures the analysis of these cell lines by immunocytochemical stainings (Oct4, Nanog, Sox2 and E cadherin) and flow cytometry (Tra 1-60, SSEA-3, H type 1, SSEA-1) demonstrated a profile characteristic for undifferentiated hESCs (FIGS. 2A and B). The expression levels of major pluripotency associated genes remained essentially similar throughout 20 passages on both matrices. Minor upregulation of primitive streak/early differentiation markers Brachyuru and Goosecoid occurred at later passages of FES29 cells on ECA (FIG. 2C). With the iPSC line HEL11.4, the pluripotency genes tended to remain higher and the differentiation genes lower on ECA throughout the culture period (FIG. 2D). In general, FES29 showed a constant gene expression pattern independent of the matrix as tested by PAHS-081 qPCR array including 84 genes controlling growth and differentiation of stem cells (FIG. 2E). The ECA cultured cells also retained their full in vivo differentiation capacity as indicated by highly complex teratoma containing all three germ layer derivatives (FIG. 2F). Both cell lines were karyotypically normal after 18 passages on ECA (not shown). Y-27632 had similar effects on the cells as pinacidil.

FIG. 2 shows A. Immunohistochemistry of hESC (FES29) and hiPSC (HEL11.4) lines cultured on ECA for 20 passages. B. FACS analysis of pluripotency-associated cell surface markers after 20 passages on ECA. C-D. Relative expression level of pluripotency associated genes (Oct4, Nanog, Sox2) and early differentiation associated genes (Brachyuru, Goosecoid) by qPCR at passage 1-20. The data were normalized against the level in cells cultured on Matrigel for the same time. Panel C: hESC (FES29); Panel D: hiPSC (HEL11.4). Error bars indicate SEM. E. PCR array analysis of pluripotency and early differentiation gene expression of cells growing either on Matrigel or on ECA at passage 9. Gene profiles were compared between FES 29 on Matrigel and on ECA. Y-axis is the intensity ratio and X-axis is the average intensity for a given gene measured on two similar HTqPCR arrays. All differences were less than two-fold. F. HE staining of a teratoma derived from FES 29 cultured on ECA 9 passages. Derivatives of all germ layers can be detected.

Next, the ability of ECA to support the growth of undifferentiated hPSC in defined cell culture medium StemPro™ was tested. Cells were first adapted to StemPro for one passage using 1:1 mix of StemPro and CM media and then only StemPro was used. The results indicated that also defined media supported self-renewal and cells maintained stem cell markers and normal karyotype detected after 9 passages on ECA in StemPro (data not shown).

### Clonogenicity Assay

Cells were dissociated with TrypLE for 5 minutes and passed through an 80-μm cell strainer (Becton Dickinson). Dissociated single cells from either ECA or Matrigel were seeded onto both ECA and Matrigel (35 cells/cm²) and cultured in mouse embryonic fibroblast-conditioned medium (CM) supplemented with 8 ng/ml basic fibroblast growth factor (bFGF). Pinacidil (100 μM) was used during passaging. To evaluate clonogenic capacity, cells were alkaline phosphatase stained and colony numbers were counted 10 days after plating.

### Cell Viability Analysis

Cells were plated and cultured on ECA and Matrigel for 6 days. Cell viability was analyzed in the beginning, on day 3 and on day 6 using Trypan Blue staining of dissociated cells. The results represent eight separate experiments, each performed in duplicate. Cell viability was tested also on plate without dissociation using Live/Dead Viability/Cytotoxity Kit (Invitrogen) according to manufacturer's instructions.

### Determination of Cell Growth Rate

FES 29 and HEL11.4 cells were passaged by collagenase IV to small clumps from ECA and Matrigel and plated on 12 well plates, approximately 6000 cells/well on both matrices. Cells were counted at two time points, day 3 and day 6.

Live cell imaging was used as an alternative method. For this purpose, FES 29 and HEL11.4 cells were dissociated by collagenase IV to small aggregates of 10-20 cells from ECA and Matrigel and plated on 12 well plates, approximately 1000 cells/well on both matrices. Cells were let to adhere in the cell culture incubator for 24 hours and the plates were then transferred into Cell IQ culture platform (CM-Technologies). All wells were imaged every second hour for five days. The images were analyzed using Cell IQ Analyzer.

### Clonogenicity and Cell Growth

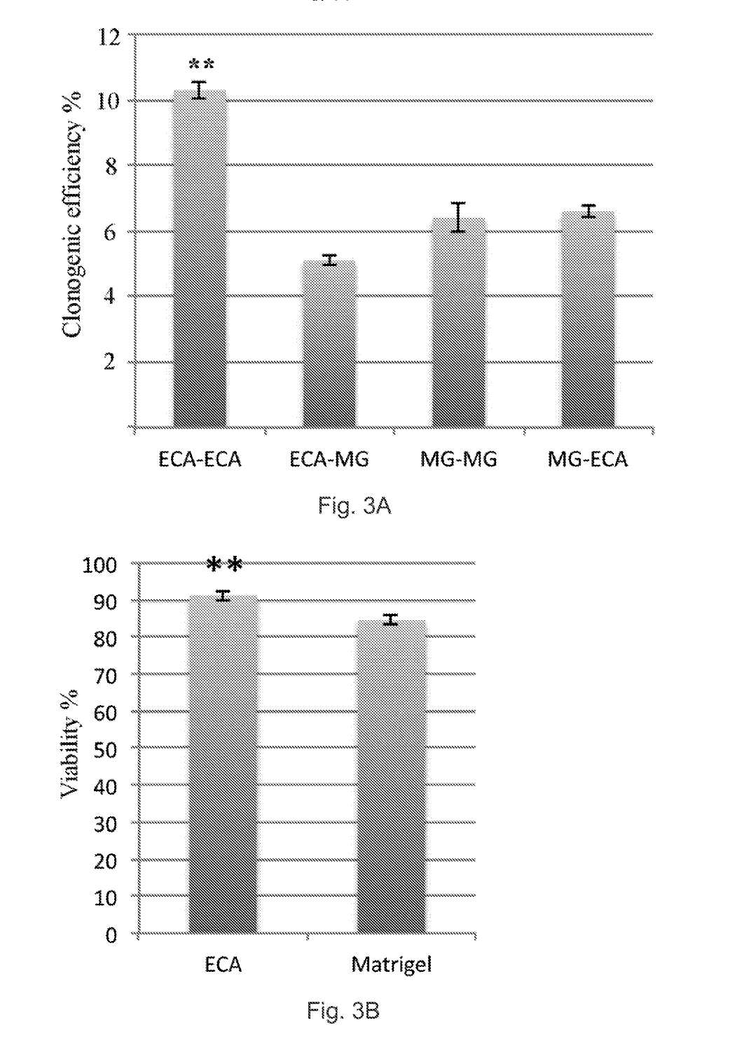

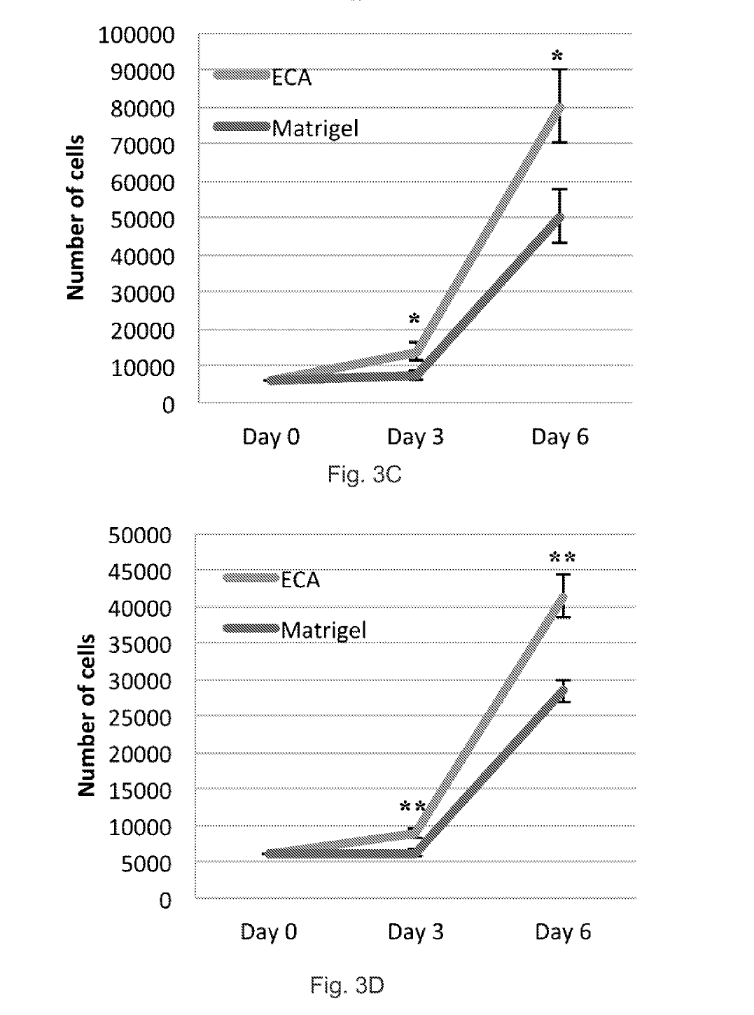

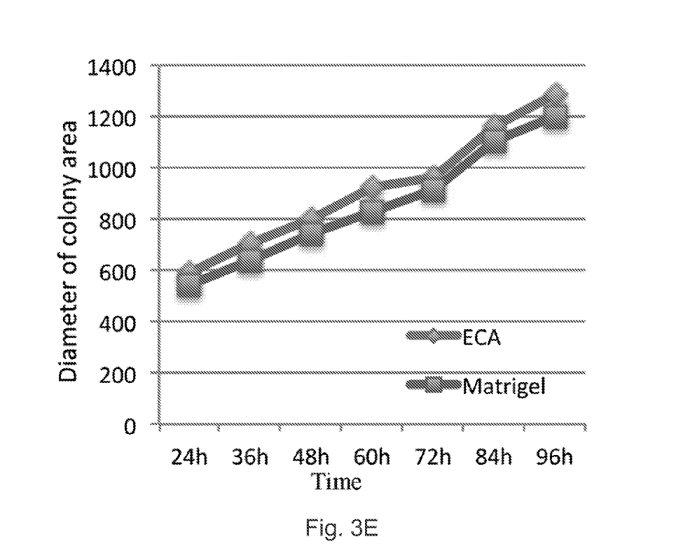

The ability of the ECA matrix to support clonogenicity of hPSC cells was studied by plating dispersed cells first adapted to ECA or Matrigel for at least two passages on either of the two matrices at the density of 35 cells/cm². In the presence of pinacidil the colony forming efficacy was clearly highest (10.3%,) when ECA-adapted cells were plated on ECA, as compared with all other conditions where the efficacy was approximately 6% (p<0.05, one-way ANOVA, Tukey's post-hoc test) (FIG. 3A). Pinacidil was found to be highly useful for the development and survival of the single-cell derived clones in these experiments.

Long-term cell imaging was used to study colony area and cell growth. Colonies were imaged every second hour during four days after plating to record colony areas and the number of cells in the colonies. In accordance with the clonogenicity assay, the initial number of colonies was higher on ECA than on Matrigel. An explanation to this was provided by cell viability analysis, which showed higher viability for cells grown on ECA than on Matrigel (90.1% vs. 82.9%, p<0.01, FIG. 3B). The cells were counted three and six days after plating. The number of cells was significantly higher on ECA than on Matrigel at both time points (FIG. 3C-D). No difference in speed of cell division was detected and the size of the colonies growing on ECA and Matrigel was similar (FIG. 3E). These results show that culture on ECA generates more cells based on better attachment and survival after dissociation and plating.

FIG. 3 shows A. Clonogenicity assay on dispersed single cells (35 cells/cm²). Cloning efficiency was calculated as the number of clones per the total number of plated cells when transferring the cells between ECA and Matrigel (MG) substrates (p=0.003, one-way ANOVA). Error bars indicate SEM. B. Cell viability during passaging. Cells were counted by trypan blue exclusion (N=8). On ECA the average cell viability rate was 92% whereas on Matrigel it was 84% (p<0.01). Data represent the mean (±SEM) of eight separate experiments. C-D. Analysis of cell growth rate. Data represent the mean) of 12 wells of hESC (FES29, C) or hiPSC (HEL11.4, D). *, p<0.05; **, p<0.01. E. Colony area (2 wells/matrix) for FES 29 cell line. Cells were passaged in small clumps and cultured in live cell imaging system (Cell IQ, CM-technologies). Areas of the colonies were analyzed in Cell IQ Analyzer program and results are shown as average size. Y-axis is the diameter of colony area counted as pixels.

### Validation of Binding Specificity

Cells were passaged and plated as described earlier. The two compounds expected to act as specific binding inhibitors for ECA were lactose monohydrate (Sigma-Aldrich, 100 mM) and lacto-N-neotetraose (Kyowa Hakko Kogyo, 100 mM). Saccharose (Sigma-Aldrich, 100 mM) was used as a control. The inhibitors were added to culture media at the time of passaging and the attached cells were counted after 20 hours.

To assess the specificity of the cell-lectin interaction in supporting stem cell attachment to the growth surface, we performed inhibition experiments with specific disaccharide inhibitors and control disaccharides. Lactose (composed of galactose β1,4-linked to glucose) inhibited cell attachment effectively at 100 mM concentration, while the same concentration of saccharose (fructose α1,1-linked to glucose) had no inhibitory activity. Further, lacto-N-neotetraose oligosaccharide, which contains the β1,4-linked galactose epitope, was as effective as lactose (p<0.001) (FIG. 4). The inhibition experiments thus demonstrated that initial cell attachment to the ECA matrix was dependent on specific interaction of the surface-bound lectin with stem cell glycan ligands. The experiments were performed either in the presence (FIG. 4A) of absence (FIG. 4B) of pinacidil. Even if the effect of the inhibitors was similar in both conditions, the total number of attached cells was 4-fold higher with pinacidil.

FIG. 4 demonstrates the validation of the binding specificity of hESC and hiPSC to ECA using specific competitive inhibitors either in the presence (A) or absence (B) of Pinacidil. The attached cells were counted 20 h after plating. Data represent the mean (±SEM) of both cell lines, FES 29 and HEL 11.4. The total number of attached cells was 2-4 fold higher when Pinacidil was used (p<0.001). The disaccharides lactose monohydrate (LacH2O) and lacto-N-neotetraose (LnNT) inhibited significantly cell attachment onto ECA (***; p<0.001). The control disaccharide, saccharose, had no inhibitory activity.

### Continued Culture

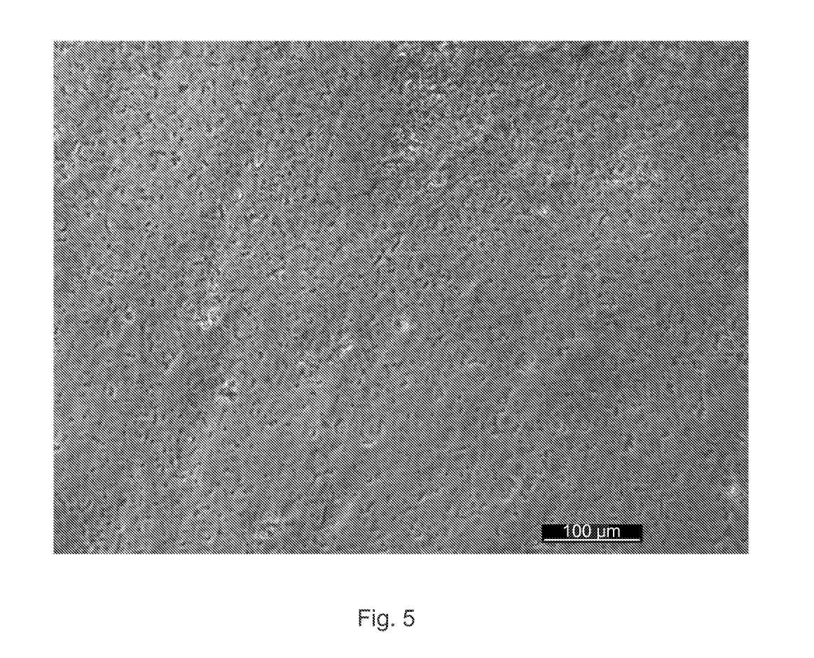

Long-term cell culture was used to study colony area and cell growth of initially formed colonies described in the previous Examples in continued culture on ECA coated surface. Colonies were imaged with microscopy to evaluate colony areas and the number of cells in the colonies. Unlike on Matrigel, the colonies continued expanding rapidly until they completely fused together and filled in various experiments from 90% to 100% of the available ECA-coated surface. FIG. 5 shows an exemplary microscopic image showing unlimited and non-colony restricted growth as a continuous cell layer over the whole available area, effectively forming a single colony. In contrast, when any two colonies grew together on Matrigel in parallel experiments, they typically did not fuse but instead the former colony border cells retained their different morphology leaving the old colony borders clearly visible in microscopic examination. On ECA, all the cells retained the morphology of pluripotent stem cells growing inside colonies: small and round cell size and shape, and high nucleus-to-cytoplasm ratio (FIG. 5). Stem cell marker expression was analyzed from these cells similarly as in Example and the results were similar (not shown). These results demonstrated that culture on ECA according to the present invention generated a different non-colony growth morphology for the cells, while the proportion of colony border cells was significantly decreased compared to Matrigel culture. In representative experiments the proportion of colony border cells as measured by microscopy was compared on Matrigel and on ECA coated surfaces in standard culture conditions with pinacidil used in passaging: after the pluripotent stem cells had essentially completely filled a well in a 6-well plate as a large colony (diameter 3.5 cm, surface area 9.5 cm²), the ratio of cells inside the colony to cells at the colony borders was over about 1000:1 (>99.9%), while in typical colonies such as on Matrigel culture the similar ratio between the phenotypically different cells was depending on the individual colony from about 10:1 (90.9%) to about 100:1 (99.0%) with much variation from experiment to experiment. Thus 10-100 fold reduction in the number of colony border cells and significantly more homogeneous cell population with significantly less variation could be achieved in pluripotent stem cell culture on ECA coated surface compared to Matrigel.

### Example 4

### Analysis of Stem Cell Culture Medium Supplements

StemPro XF supplement, KO serum replacement and ITS supplement were purchased from Invitrogen (Life Technologies) and subjected to N-glycosidase F digestion (Nyman, T. A., et al., 1998. Eur. J. Biochem. 253:485-93). Sialylated N-glycans were isolated by graphitized carbon microcolumn solid-phase extraction (Hemmoranta, H., et al., 2007. Exp. Hematol. 35:1279-92) and analyzed by MALDI-TOF mass spectrometry in negative ion linear mode (Heiskanen, A., et al., 2009. Glycoconj. J. 26:367-84). Neutral N-glycan fraction was similarly analyzed in positive ion reflector mode.

In the supplements, neutral N-glycans were not detected and were thus below the detection limit of approximately 1% of the total N-glycans. Glycan signals indicating the presence of Neu5Gc (Heiskanen, A., et al., 2007. Stem Cells 25:197-202) including m/z 1946, 2237, and 2256, were not detected in XF supplement, while the major acidic N-glycan components were S2H5N4, S1H5N4, S2H6N5F2, S2H5N4F1 and S2H6N5 (for nomenclature see Heiskanen, A., et al., 2007. Stem Cells 25:197-202), together comprising over 90% of the total detected glycan signals. This corresponds to a typical human transferrin N-glycan composition of partially sialylated biantennary and triantennary N-glycans that contain non-reducing terminal N-acetyllactosamine at the antennae (Spik, G., et al., 1988. Biochimie 70:1459-69). Similarly, the major acidic N-glycan components in KO supplement were S2H5N4, S1G1H5N4, G2H5N4, S1H5N4, and G1H5N4, together comprising over 90% of the total detected glycan signals. This corresponds to a typical bovine serum transferrin N-glycan profile with presence of Neu5Gc (Heiskanen, A., et al., 2007. Stem Cells 25:197-202). ITS supplement gave essentially similar results as KO serum replacement.

Calculation of approximate number of non-reducing terminal Galβ1-4GlcNAc residues in an N-glycan site (nLN) was performed according to the formula:

$$nLN = {\sum }_{i = 1}^{x} [ \% Gi ( N - 2 - S ) ] ,$$

wherein x is the total number of detected glycan signals, % Gi is the proportion of glycan Gi of the total detected glycans, N is the number of N-acetylhexosamine residues in the proposed glycan composition, S is the number of Neu5Ac and Neu5Gc residues in the proposed glycan composition, N-2 is the number of antennae in glycan Gi, and N-2-S is the maximal number of non-reducing terminal Galβ1-4GlcNAc antennae in glycan Gi. This calculation yielded that nLN=0.37 for XF supplement and nLN=0.24 for KO supplement. However, nLNmax can be up to 2 in case the biantennary N-glycans are essentially completely desialylated (asialotransferrin).

Transferrin is the major glycoprotein added to the Invitrogen's serum-free medium supplements XF, KO and ITS. According to the manufacturer's specifications, final transferrin amounts used in cell culture media can vary between 0.5 mg/l to 100 mg/l in promoting the growth of both adherent and suspension cell cultures (reference cited: Barnes, D., and Sato, G., 1980. Anal. Biochem. 102:255-70). From these figures effective inhibiting oligosaccharide structure concentrations originating from transferrin-containing supplements can be calculated. For example, using XF supplement containing human serum transferrin with two N-glycosylation sites and a molecular weight of about 80 kg/mol, N-glycan concentration is 25 nmol/mg; and effective inhibiting oligosaccharide structure concentration is thus from 5 nM (0.5 mg/l) up to about 1 μM (100 mg/l) for supplemented human transferrin of nLN=0.37. With an nLNmax=2 in the case of asialotransferrin, the concentration is from 25 nM (0.5 mg/l) up to about 5 μM (100 mg/l); and using KO supplement containing bovine serum transferrin with one N-glycosylation site and a molecular weight of about 80 kg/mol, N-glycan concentration is 13 nmol/mg; and effective inhibiting oligosaccharide structure concentration is thus from about 1.5 nM (0.5 mg/l) up to about 0.3 μM (100 mg/l) for supplemented bovine transferrin of nLN=0.24. With an nLNmax=2 in the case of asialotransferrin, the concentration is from about 13 nM (0.5 mg/l) up to about 2.5 μM (100 mg/l). As demonstrated in the above Examples, these final medium concentrations of an inhibiting oligosaccharide structure are not significantly inhibitory to stem cell culture with a carbohydrate-binding protein in the presence of a Rho-associated kinase inhibitor.

### Example 5

### Isolation and Characterization of Fully Reprogrammed Human iPS Cells

Human foreskin fibroblasts (HFF cell line CRL-2429; ATCC, Manassas, Va., USA) were reprogrammed by using retrovirus-induced overexpression of four genes: OCT4, SOX2, KLF4, and c-Myc (OSKM). Cells were infected with virus-containing supernatant twice at 24-h intervals. The infected cells were frozen three days after infection. Thawed HFF/OSKM iPS cells were plated on 6 well Matrigel (BD) coated plate (appr. 75 000 cells/plate) and cultured with hES medium up to day 10-15 after infection for FACS sorting (or up to day 22 for manual colony picking). Cells were harvested by Tryple to obtain single cell suspension and sorted by FACS using antibodies against Tra-1-60 (Millipore) and Lewis c blood group antigen Galβ1-3GlcNAcβ (clone K21). Target cell populations (Tra+Lewis+, Tra+Lewis−, Tra-Lewis+, Tra-Lewis−, Tra+, and Lewis+) were sorted directly onto Matrigel and cultured in E8 medium (Life Technologies) supplemented with ROCK inhibitor pinacidil (Sigma-Aldrich). 24 hours after sorting pinacidil was removed from the medium. As a control, formed colonies were manually picked onto Matrigel in E8 medium (supplemented 24 hours with pinacidil). Alternatively, ECA lectin matrix (Sigma-Aldrich) was used instead of Matrigel. Both the manually picked and FACS-sorted iPS cells were passaged with 0.5 mM EDTA from passage 1. Cells were cultured up to passage 10 for characterization.

Immunostaining of reprogrammed fibroblasts at time points Day 10 and Day 13 was performed. At Day 10, clusters of Tra-1-60 and Lewis c positive cells could be detected by immunofluorescence imaging. At Day 15, they had matured into visible but small colonies. The colonies were mosaics of Tra+Lewis+, Tra+Lewis−, Tra-Lewis+ and Tra-Lewis− cells.

FACS sorting and derivation of iPSCs at time points Day 10, Day 13 and Day 15 after infection was performed. The proportion of both Tra+ and Lewis+ cells increased drastically during reprogramming between Days 10 and 13. Lewis+ cells appeared slightly earlier (Day 10) than Tra+ cells. Both Tra+Lewis+ and Tra-Lewis+ cells formed iPSC colonies after sorting. This showed that reprogrammed cells were present already at Day 10. In contrast to Lewis+ cells, Tra+Lewis− cells sometimes contained a small proportion of differentiated cells. Tra-Lewis− cells produced no colonies in the experiments. Relative proportions of Tra+Lewis−, Tra+Lewis+ and Tra-Lewis+ cells were 0%, 0.2% and 0.5% at Day 10; 0.5%, 9.5% and 3.5% at Day 13; and 0.1%, 9.7% and 3.4% at Day 15; respectively.

When the infected cells were FACS-sorted and either Tra+Lewis+, Tra-Lewis+, or Lewis+ cells cultured further, they formed morphologically stable iPS cell colonies that were free of differentiated cells and had significant cell mass after second passage (between Day 24 and Day 30 after infection). In contrast, manually picked colonies were initially (at Day 22 after infection) mixtures of iPSCs, incompletely reprogrammed cells and differentiated cells, and formed morphologically stable colonies only after 5-10 passages at between Day 50 and Day 70 after infection.

FACS sorting derived iPSC lines generated compact, morphologically normal colonies which expressed common markers of pluripotency. No differences to the manually picked and further cultured iPSC lines were detected with regard to marker expression. The FACS sorted cell lines were characterized at passage 10: they were positive for cell surface markers Tra-1-60, SSEA-3 and H type 1, as well as gene expression markers OCT-4, NANOG, SOX2 AND TGFβ1.

### Example 6

### Generation of iPS Cells Using Episomal Sendai Virus or Non-Integrating Vectors

CytoTune® iPS 2.0 Sendai Reprogramming Kit is purchased from Life Technologies. The kit contains the reprogramming vectors Oct, Sox2, Klf4, and c-Myc. The human fibroblast cells (on feeder cells/vitronectin) are reprogrammed according to the Kit's instructions.

The following presents the reprogramming timeline (according to the Kit):

Days −2 to day 6: Preparation of human fibroblasts for transduction, transduction of the cells using the CytoTune® 2.0 Sendai reprogramming vectors, replacement of the medium and preparation of MEF/vitronectin culture dishes.

Days 7 and 8: Plating transduced cells on MEF culture dishes or vitronectin coated culture dishes and change the medium to iPSC medium or complete Essential 8™ Medium, respectively.

Days 9-28: Replacement of medium and monitoring the culture vessels for the emergence of iPSC colonies. For BJ fibroblasts, colony formation may be observed on Day 12, but depending on the cell type, culturing for up to 4 weeks before seeing colonies is possible.

Peripheral blood mononuclear cells (PBMCs; on MEFs) or CD34+ cells are induced according to Kit's instructions, the reprogramming timeline for PBMCs and CD34+ is similar to fibroblasts as above. By day 15 to 20/21 after transduction, colonies should have grown to an appropriate size for transfer.

It takes about 1-2 months after gene transduction to obtain iPSCs free of CytoTune® 2.0 Sendai reprogramming vectors.

On each day of post induction, K21 and/or Tra-1-60 FACS selection may be performed for iPSCs as described above in Examples 1 or 5. K21⁺ and/or K21⁺/Tra-1-60⁺ cells are selected and cultured to form homogenous iPS cell colonies whereas K21⁻ cells (whether negative or positive for Tra-1-60) are used as controls. K21⁺ and/or K21⁺/Tra-1-60⁺ cells may appear as early as day 1, day 2, day 3, day 4, day 5, day 6, day 7, day 8, day 9, day 10, day 11, day 12, day 13, day 14, or day 15 post induction. K21⁺ and/or K21⁺/Tra-1-60⁺ cells are selected directly to 96 or 384 well plates, one iPSC per one well or 2-10 iPSCs per one well, or to 24 well plates, 100-500 iPSC per one well or about 500 or 1000-2000 or 2000-5000 iPSC per one well.

### Induction of Non-Pluripotent Cells by Episomal Vectors

Epi5™ Episomal iPSC Reprogramming Kit is purchased from Life Technologies. The kit contains (Oct3/4, Sox2, Klf4, L-myc, and Lin28), and plasmids that express the p53 dominant negative mutant (mp53DD) and EBNA1 genes. The human fibroblast cells or CD34⁺ cells are reprogrammed according to the Kit's instructions. Days 15 to day 21 are used to observe the emergence of iPSC colonies.

On each day of post induction, K21 and Tra-1-60 FACS selection is performed for iPSCs as described above in Examples or 5. K21⁺ and/or K21⁺/Tra-1-60⁺ cells are selected and cultured to form homogenous iPS cell colonies whereas K21⁻ cells (whether negative or positive for Tra-1-60) are used as controls. K21⁺ and/or K21⁺/Tra-1-60⁺ cells may appear as early as day 1, day 2, day 3, day 4, day 5, day 6, day 7, day 8, day 9, day 10, day 11, day 12, day 13, day 14, or day 15 post induction. K21⁺ and/or K21⁺/Tra-1-60⁺ cells are selected directly to 96 or 384 well plates, one iPSC per one well or 2-10 iPSCs per one well, or to 24 well plates, 100-500 iPSC per one well or about 500 or 1000-2000 or 2000-5000 iPSC per one well.

Induction of Non-Pluripotent Cells by Synthetic mRNA

Stemgent® mRNA Reprogramming Kit is purchased from Miltenyi Biotech (Catalogue number 00-0071). The kit contains modified mRNAs encoding Oct4, Klf4, Sox2, c-Myc, Lin-28, and nuclear GFP (nGFP) as well as B18R recombinant protein, which reduces the cellular interferon response to exogenous mRNAs. The human fibroblast cells or other non-pluripotent cells are reprogrammed according to the Kit's instructions or according to Warren et al. (2012) Feeder-free derivation of human induced pluripotent stem cells with messenger RNA. Scientific Report, 2:657, DOI: 10.1038/srep00657, or Warren et al. (2010) Highly efficient reprogramming to pluripotency and directed differentiation of human cells using synthetic modified mRNA, Cell Stem Cell. 7: 618-630. doi:10.1016/j.stem.2010.08.012.

Transfection of non-pluripotent cells may take up to 17 days and first iPSCs may be observed from day 18.

On each day of post induction, K21 and Tra-1-60 FACS selection is performed for iPSCs as described above in Examples or 5. K21⁺ and/or K21⁺/Tra-1-60⁺ cells are selected and cultured to form homogenous iPS cell colonies whereas K21⁻ cells (whether negative or positive for Tra-1-60) are used as controls. K21⁺ and/or K21⁺/Tra-1-60⁺ cells may appear as early as day 1, day 2, day 3, day 4, day 5, day 6, day 7, day 8, day 9, day 10, day 11, day 12, day 13, day 14, or day 15 post induction. K21⁺ and/or K21⁺/Tra-1-60⁺ cells are selected directly to 96 or 384 well plates, one iPSC per one well or 2-10 iPSCs per one well, or to 24 well plates, 100-500 iPSC per one well or about 500 or 1000-2000 or 2000-5000 iPSC per one well.

### Example 7

### Generation of iPS Cells from Blood

Peripheral blood mononuclear cells (PBMCs) are isolated from peripheral blood using Vacutainer CPT tubes (BD Biosciences) according to manufacturer's instructions, washed with PBS and suspended in PBS. For reprogramming, the cells are transferred to suitable medium, for example RPMI-1640 medium supplemented with 10% fetal calf serum or appropriate amount of serum supplement at 10⁶ cells/ml, or to a xeno-free medium selected from E6 medium (Life Technologies) and Nutristem XF/FF culture medium (Stemgent), plated on Matrigel (for E6 medium) or on xeno-free substrate selected from laminin-521 (Biolamina; for Nutristem medium) and vitronectin (Life Technologies; for E6 medium), and reprogrammed as described in the preceding Examples and 6. Reprogrammed pluripotent stem cells are isolated by FACS as above using antibodies against Tra-1-60 (Millipore) and Lewis c blood group antigen Galβ1-3GlcNAcβ (clone K21), or xeno-free Tra-1-60 antibody and xeno-free K21 or xeno-free anti-PLN antibody for xeno-free FACS isolation. Target cell populations (Tra-1-60+/K21+ and K21+) are sorted and cultured on Matrigel in E8 medium (Life Technologies) or Nutristem XF/FF culture medium (Stemgent) supplemented with ROCK inhibitor pinacidil (Sigma-Aldrich) as described above, or for xeno-free culture plated on xeno-free substrate selected from laminin-521 (Biolamina; for Nutristem medium) and vitronectin (Life Technologies; for E6 medium). Sorted and cultured cells form morphologically stable iPS cell colonies which express common markers of pluripotency: Tra-1-60, SSEA-3 and H type 1, as well as gene expression markers OCT-4, NANOG, SOX2 AND TGFβ1. iPS cell cultures contacted in each step of the process with only xeno-free reagents as described above generate xeno-free iPS cell lines.

### Example 8

### Determination of Relative iPS Cell Colony Forming Efficiency of Differently Sorted Reprogrammed Cells

Human foreskin fibroblasts were reprogrammed with retroviral vectors as described above and subjected to FACS sorting after induction and culturing on day 15 to determine relative colony-forming efficiency of different cell populations. K21+, Tra-1-60+, K21+Tra-1-60+ and K21-Tra-1-60− cells were sorted 500 cells/one well in 24-well plate and cultured as described above. At least 12 experiments were performed with each cell population and the number of individual pluripotent colonies that were formed after several days culture, were counted. K21-Tra-1-60− double-negative cells did not form any colonies. K21+Tra-1-60+ double-positive cells were most efficient in forming pluripotent cell colonies and formed 1 colony per 750 sorted and plated cells. K21+ single-positive and Tra-1-60+ single-positive cells were less efficient in forming pluripotent cell colonies and formed 1 colony per 1333 sorted and plated cells and 1 colony per 2000 sorted and plated cells, respectively.

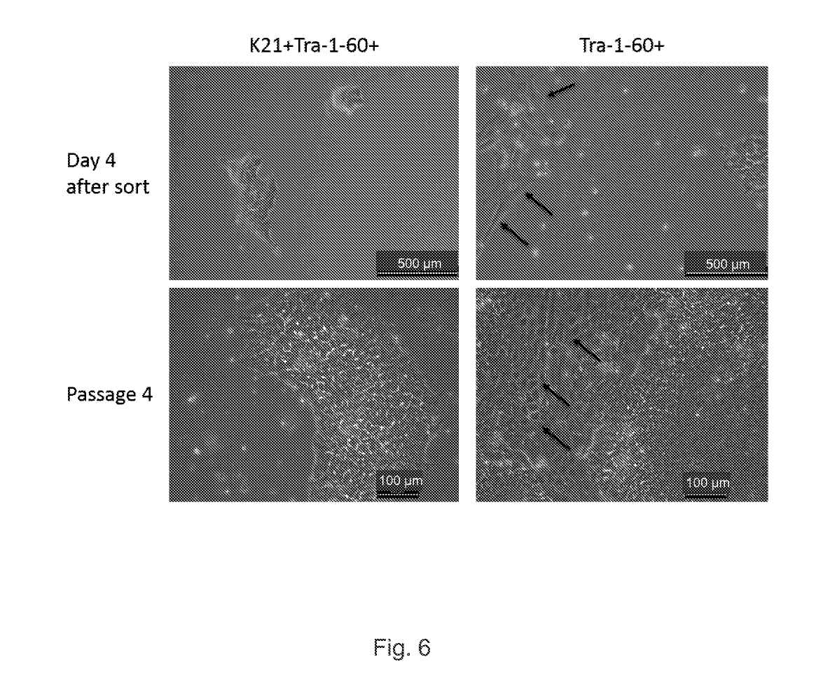

FIG. 6 shows a comparison of reprogrammed and FACS sorted Tra-1-60+/K21+ double positive cells (left panels) and Tra-1-60+ single positive cells (right panels) at four days (upper panels) and four passages (lower panels) after sorting. Both cell lines produced iPS cell colonies with characteristic cell morphology in culture, but only cells selected with the antibody K21 during FACS sorting were free of contaminating differentiated cells. Arrows point to differentiated cells present among Tra-1-60+ single positive cells but not among Tra-1-60+/K21+ double positive cells. At passage 4, the space left to the Tra-1-60+ single positive cell colony is filled with differentiated cells.

By microscopic observation from the experiment series, it was thus determined that no contaminating differentiated cells were present in the wells to which the K21+Tra-1-60+ double-positive cells were sorted. In contrast, contaminating differentiated cells were present in the wells to which the Tra-1-60+ single-positive cells were sorted. In conclusion, this demonstrated that sorting K21+Tra-1-60+ double-positive cells was effective in producing pure iPS cell cultures in the FACS sorting step and that sorted Tra-1-60+ single-positive cells contained differentiated cells as contaminant. Also in K21+ single-positive cells no contaminating cells were observed.

### Example 9

### Generation of iPS Cells by Sorting with Anti-H Type 1 Antibody

Fibroblasts: Human foreskin fibroblasts were retrovirally reprogrammed as described above and 17-206+ cells and 17-206+ cell colonies were identified by live cell imaging after induction and culturing with fluorescently labelled anti-H type 1 antibody 17-206 (Abcam), which binds to non-reducing terminal saccharide sequence Fucα1-2Galβ1-3GlcNAcβ, as well as fluorescently labelled Tra-1-60 antibody (Millipore). 17-206+ cells colocalized together with Tra-1-60 in both individual reprogrammed cells that emerged after reprogramming and the forming iPS cell colonies starting from about day 10. Thus anti-H type 1 antibody 17-206 staining could be used for directing both manual colony picking and FACS sorting to generate new iPS cell lines from human fibroblasts.

Blood cells: Mononuclear human blood cells are reprogrammed with Sendai virus vectors as described above and 17-206+ cells are isolated by FACS sorting after induction and culturing with anti-H type 1 antibody 17-206 (Abcam), which binds to non-reducing terminal saccharide sequence Fucα1-2Galβ1-3GlcNAcβ, 500 cells/one well in 24-well plate and cultured as described above. The sorted 17-206+ cells form morphologically stable iPS cell colonies which express common markers of pluripotency: Tra-1-60, SSEA-3 and H type 1, as well as gene expression markers OCT-4, NANOG, SOX2 AND TGFβ1.

### Example 10

### Sequencing of Mouse Antibody Clone K21

K21 cells were cultivated over three passages. Total RNA was purified from 10×10⁶ cells with Fermentas GeneJet RNA purification kit according to manufacturer's instructions. 10 μg of Purified total RNA was used for RACE (Rapid Amplification of cDNA Ends), made with Epicentre ExactSTART Eukaryotic mRNA 5′- & 3′-RACE Kit. During the procedure, uncapped mRNA molecules were selected out by converting them to nonligatable 5′ hydroxyl RNA, by treatment with heat-labile Apex alkaline phosphatase. 5′ capped mRNA's were then enriched by removing the cap with Tobacco Acid Pyrophosphatase and tagging the 5′ end with 5′-RACE acceptor oligo with PCR priming site to kit's primer 1. For subsequent cDNA synthesis, gene-specific primers GP927 (IgM kappa light chain constant sequence) and GP922 (IgM heavy chain constant sequence) were used instead of kit's oligo (dT). Specific cDNA sequences were then PCR amplified by using kit's Primer 1 as 5′ primer and gene specific primers GP927 or GP922.

PCR amplified sequences were phosphorylated and cloned into pUC18 backbone opened with restriction endonuclease SmaI. Insert-containing clones were screened by PCR made with pUC universal/reverse primers. In the case of heavy chain, screening was enhanced by using a nested primer GP921. PCR-positive clones were sequenced with pUC universal- or reverse primer. In total, 15 clones for LC and 30 clones for HC were sequenced.

TABLE 3
Primers used for amplifying specific cDNA
sequences.

| | |
|---|---|
| Primer 1 | TCATACACATACGATTTAGGTGACACTATAGAGCGGCCGC |
| | CTGCAGGAAA (SEQ ID NO: 37) |
| GP921 | GACATTTGGGAAGGACTGACTCTG (SEQ ID NO: 38) |
| GP922 | CTGAACCTTCAAGGATGCTC (SEQ ID NO: 39) |
| GP927 | GCTGTAGGTGCTGTCTTTGC (SEQ ID NO: 40) |

From 15 light chain clones two distinct light chain sequences were identified and from 30 clones of heavy chain one heavy chain sequence was identified. In order to produce full-length recombinant anti-PLN antibodies the obtained partial IgM light chain and heavy chain mouse sequences were C-terminally complemented with corresponding mouse or human antibody sequences (mouse IgM kappa light chain complemented from light chain of racotumomab, ref 8998 in IMGT database; mouse IgM heavy chain complemented from or replaced by heavy chain of racotumomab, ref 8998 in IMGT dataset or herceptin antibody, respectively).

The cloning of full-length anti-PLN antibodies may be performed as described below. Based on SEQ ID NO:s 13-20, cDNA sequences for open reading frames are generated based on codon usage of the organism to be used to express and secrete the antibodies (for example CHO cells). The full-length light and heavy chains are artificially synthesized, the light and heavy chains are cloned into appropriate mammalian expression vectors and transfected into e.g. CHO cells. IgM or IgG antibodies are produced in CHO cells, secreted into the culture medium and purified using methods known for skilled artisan. Alternatively, if endogenous, native K21 IgM heavy and light chains sequences are desired, further RACE cloning can be performed. Alternatively, if desirable, mouse light and heavy chain variable CDR regions can be grafted into e.g. human variable region framework using methods known for skilled artisan. Mouse kappa light chain can also be replaced into human kappa light chain.

Xeno-free anti-PLN antibodies are generated by transfecting e.g. GMP certified CHO cells by the above light chain and heavy chain sequences, the CHO cells are cultured in xeno-free, defined medium, and IgG or IgM antibodies are purified from culture medium using methods known for skilled artisan. If IgM heavy chain is expressed, for example, in CHO cells, see Mader et al. (2013), Recombinant IgM expression in mammalian cells: A target protein challenging biotechnological production, Advances in Bioscience and Biotechnology, 2013, 4, 38-43, doi:10.4236/abb.2013.44A006; Wolbank et al. (2003) Characterization of human class-switched polymeric (immunoglobulin M [IgM] and IgA) anti-human immunodeficiency virus type 1 antibodies 2F5 and 2G12. J Virol. 77:4095-4103; Tchoudakova et al. 2009 High level expression of functional human IgMs in human PER.C6 cells. MAbs. 1:163-171; and Chromikova et al. (2014) Evaluating the bottlenecks of recombinant IgM production in mammalian cells, Cytotechnology, epub, DOI 10.1007/s10616-014-9693-4. In full-length IgM heavyn chain molecules two C-terminal cysteines may be deleted if non-pentameric or non-hexameric IgM molecules are desired to be produced.

### Example 11

### Comparison of FACS Sorted and Manually Picked Reprogrammed Cells

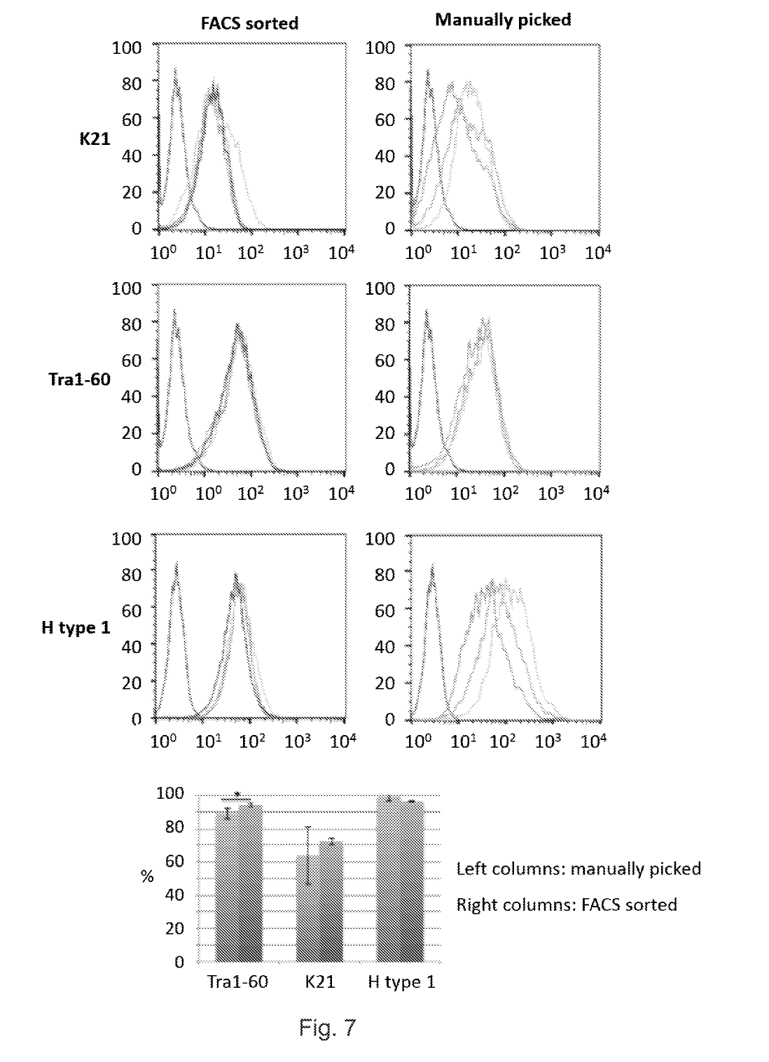

Human foreskin fibroblasts were retrovirally reprogrammed as described above. Multiple iPS cell lines were derived by either 1) FACS sorting of Tra-1-60+/K21+ double-positive cells on day 15 after induction, or 2) manual picking of emerged colonies after 20 days after induction. After cell line derivation, they were cultured for 5 passages and analysed expression of the cell surface markers K21 (Abcam), Tra-1-60 (Millipore) and H type 1 (clone 17-206, Abcam). Three cell lines were analyzed (three curves in each panel between 10⁰-10⁴ fluorescence intensity) and compared to control without marker antibody (one curve in each panel between 10⁰-10¹ fluorescence intensity). The results are shown in FIG. 7, which demonstrates the analysis of iPS cell lines derived from retrovirally reprogrammed fibroblasts either by FACS sorting of Tra-1-60+/K21+ double positive cells or by manual picking of emerged colonies. After cell line derivation, they were cultured for 5 passages and analyzed. The six panels show FACS analysis results of cell surface marker expression of K21 (upper panels), Tra-1-(middle panels) and H type 1 (lower panels). Three cell lines were analyzed (three curves in each panel between 10⁰-10⁴ fluorescence intensity) and compared to control without marker antibody (one curve in each panel between 10⁰-10¹ fluorescence intensity). FACS sorted iPS cell lines (left panels) had homogeneous expression of the markers in each cell line, while manually picked iPS cell lines (right panels) had more heterogeneous expression of the markers, showing that even after prolonged culture and passaging the manually picked iPS cell lines were still more heterogeneous than the FACS sorted cell lines.

As is clear for a person skilled in the art, the invention is not limited to the examples and embodiments described above, but the embodiments can freely vary within the scope of the claims.

1.-47. (canceled)

48. A method for generating induced pluripotent stem cells, wherein the method comprises:
step a) of inducing non-pluripotent cells to produce a mixture comprising induced pluripotent stem cells and non-pluripotent cells;
step b) of contacting the mixture comprising induced pluripotent stem cells and non-pluripotent cells obtainable from step a) with a binding agent,
wherein the binding agent is capable of binding an epitope consisting of the non-reducing terminal saccharide structure according to formula
(Fucα1-2)ₐGalβ1-3HexNAcβ,
wherein a=0 or 1 and Hex is either Glc or Gal; and
step c) of selecting an induced pluripotent stem cell bound by the binding agent.

49. The method according to claim 48, wherein the binding agent is capable of binding an epitope consisting of the non-reducing terminal disaccharide structure according to formula
Galβ1-3HexNAcβ,
or trisaccharide structure according to formula
Fucα1-2Galβ1-3HexNAcβ,
wherein Hex is either Glc or Gal.

50. The method according to claim 48, wherein the binding agent is an antibody or a lectin.

51. The method according to claim 50, wherein the antibody is K21, a xeno-free K21, anti-PLN, a xeno-free anti-PLN, Hesca-2, a xeno-free Hesca-2, A68-E/E3, a xeno-free A68-E/E3, A68-E/A2, a xeno-free A68-E/A2, A68-B/A11, a xeno-free A68-B/A11, 17-206, a xeno-free 17-206, anti-SSEA-5, a xeno-free anti-SSEA5, A70-A/A9, a xeno-free A70-A/A9, A51-B/A6, a xeno-free A51-B/A6, MBr1 or a xeno-free MBr1, or wherein the lectin is peanut agglutinin, Bauhinea purpurea lectin (BPL), Maclura pomifera lectin (MPL), Sophora japonica lectin (SJL), Artocarpus lakoocha (Artocarpin) lectins or Abrus precatorius agglutinin (APA).

52. The method according to claim 48, wherein an induced pluripotent stem cell bound by the binding agent is selected and isolated using a method of cell separation such as fluorescent-activated cell sorting (FACS), magnetic-activated cell sorting or affinity chromatography.

53. The method according to claim 48, wherein step a) comprises inducing non-pluripotent cells to produce a mixture comprising induced pluripotent stem cells and non-pluripotent cells and cultivating the mixture comprising induced pluripotent stem cells and non-pluripotent cells obtained.

54. The method according to claim 53, wherein the mixture comprising induced pluripotent stem cells and non-pluripotent cells is cultivated for a time period.

55. The method according to claim 48, wherein step b) further comprises contacting the mixture comprising induced pluripotent stem cells and non-pluripotent cells obtainable from step a) with a secondary binding agent or a xeno-free secondary binding agent capable of binding a secondary epitope; and step c) comprises selecting an induced pluripotent stem cell bound by the binding agent and by the secondary binding agent or the xeno-free secondary binding agent capable of binding the secondary epitope.

56. The method according to claim 55, wherein the secondary epitope is selected from the group consisting of Tra-1-60, Tra-1-81, Tra-2-54, SSEA-3, SSEA-4, H type 1, Lewis y, alkaline phosphatase, CD9, CD24, CD29, CD30, CD44, CD49c, CD49f, CD50, CD51/61, CD56, CD57, CD58, CD71, CD73, CD98, CD105, CD117, CD133, CD140a, CD146, CD193, CD196, CD271, CD309, CD326, CD338, GCTM-2, TG30, and TG343, or wherein the secondary binding agent is the antibody Tra-1-60 or a xeno-free Tra-1-60 antibody.

57. The method according to claim 48, wherein a population of cells bound by the binding agent is selected in step c); and wherein at least 80%, or at least 90%, or at least 95%, or at least 97%, or at least 98%, or at least 99%, or at least 99.5%, or 100% of the cells of the population selected in step c) are induced pluripotent stem cells.

58. The method according to claim 48, wherein the method comprises:
step d) of cultivating the induced pluripotent stem cell obtainable from step c).

59. The method according to claim 58, wherein the induced pluripotent stem cell obtainable from step c) and/or the mixture comprising induced pluripotent stem cells and non-pluripotent cells obtained in step a) are cultivated in step d) and/or in step a) in a culture essentially free of feeder cells and/or in xeno-free culture medium.

60. The method according to claim 58, wherein non-pluripotent cells are induced in step a) using extra-chromosomal programming elements; and wherein the extra-chromosomal programming elements are not present in the induced pluripotent stem cell obtainable from step c) and/or step d).

61. The method according to claim 58, wherein the induced pluripotent stem cell obtainable from step c) and/or the mixture comprising induced pluripotent stem cells and non-pluripotent cells obtained in step a) is contacted with a carbohydrate-binding protein during the cultivation in step d) and/or in step a),
wherein the carbohydrate-binding protein is capable of binding the non-reducing terminal oligosaccharide structure according to the formula
(Fucα1-2)ₙGalβ1-4GlcNAc,
wherein n=0 or 1.

62. The method according to claim 58, wherein the induced pluripotent stem cell obtainable from step c) and/or the mixture comprising induced pluripotent stem cells and non-pluripotent cells obtained in step a) is contacted with the carbohydrate-binding protein and a Rho-associated kinase inhibitor simultaneously at one or more time intervals during the cultivation in step d) and/or in step a).

63. A composition comprising the induced pluripotent stem cell or the induced pluripotent stem cell population obtainable by the method according to claim 48.

64. A culture system for generating induced pluripotent stem cells, wherein the culture system comprises a culture vessel, a culture medium, a binding agent, and optionally a carbohydrate-binding protein, wherein
the binding agent is capable of binding an epitope consisting of the non-reducing terminal saccharide structure according to formula
(Fucα1-2)ₐGalβ1-3HexNAcβ,
wherein a=0 or 1 and Hex is either Glc or Gal; and
the carbohydrate-binding protein is capable of binding the non-reducing terminal oligosaccharide structure according to the formula
(Fucα1-2)ₙGalβ1-4GlcNAc,
wherein n=0 or 1.

65. The culture system according to claim 64, wherein the binding agent is an antibody or a lectin.

66. The culture system according to claim 64, wherein the antibody is K21, anti-PLN, Hesca-2, A68-E/E3, A68-E/A2, A68-B/A11, 17-206, anti-SSEA-5, A70-A/A9, A51-B/A6 or MBr1, or wherein the lectin is peanut agglutinin, Bauhinea purpurea lectin (BPL), Maclura pomifera lectin (MPL), Sophora japonica lectin (SJL), Artocarpus lakoocha (Artocarpin) lectins or Abrus precatorius agglutinin (APA).

67. The culture system according to claim 64, wherein the culture system further comprises an apparatus for handling, culturing and/or dispensing non-pluripotent cells, a mixture of cells comprising induced pluripotent cells and non-pluripotent cells and/or selected induced pluripotent stem cells, such as a liquid handler robot and/or an incubator; an apparatus for selecting induced pluripotent stem cells, such as an apparatus for fluorescent-activated cell sorting (FACS), magnetic-activated cell sorting or affinity chromatography; and/or means for mechanical or chemical separation of induced pluripotent stem cells.


SEQUENCE LISTING (40 sequences)

SEQ ID NO: 1 - protein, 7 aa, Artificial Sequence
  HCDR1
FTFSDFY

SEQ ID NO: 2 - protein, 10 aa, Artificial Sequence
  HCDR2
SRNKAKDYTT

SEQ ID NO: 3 - protein, 9 aa, Artificial Sequence
  HCDR3
DGLLEAMDY

SEQ ID NO: 4 - protein, 16 aa, Artificial Sequence
  LCDR1 A
RSSQSLVHSNGNTYLH

SEQ ID NO: 5 - protein, 7 aa, Artificial Sequence
  LCDR2 A
KVSNRFS

SEQ ID NO: 6 - protein, 9 aa, Artificial Sequence
  LCDR3 A
SQSTHVPWT

SEQ ID NO: 7 - protein, 11 aa, Artificial Sequence
  LCDR1 B
KASENVVTYVS

SEQ ID NO: 8 - protein, 7 aa, Artificial Sequence
  LCDR2 B
GASNRYT

SEQ ID NO: 9 - protein, 9 aa, Artificial Sequence
  LCDR3 B
GQGYSYPYT

SEQ ID NO: 10 - protein, 118 aa, Artificial Sequence
  Heavy chain, variable
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTV

SEQ ID NO: 11 - protein, 114 aa, Artificial Sequence
  Light chain A, variable
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKRA

SEQ ID NO: 12 - protein, 108 aa, Artificial Sequence
  Light chain B, variable
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKR

SEQ ID NO: 13 - protein, 219 aa, Artificial Sequence
  Light chain A, full-length
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 14 - protein, 214 aa, Artificial Sequence
  Light chain B, full-length
NIVMTQSPKSMSMSVGERVTLTCKASENVVTYVSWYQQKPEQSPKLLIYGASNRYTGVPDRFTGSGSATDFTLTISSVQAEDLADYHCGQGYSYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 15 - protein, 450 aa, Artificial Sequence
  Heavy chain1 mouse IgM variable, mouse IgM CH1, mouse IgG1 hinge, mouse IgG1 CH2, mouse IgG1 CH3
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 16 - protein, 445 aa, Artificial Sequence
  Heavy chain2 mouse IgM variable, mouse IgG1 CH1, mouse IgG1 hinge, mouse IgG1 CH2, mouse IgG1 CH3
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSEAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 17 - protein, 575 aa, Artificial Sequence
  Heavy chain3 mouse IgM, full length (mouse IgM variable to IgM4)
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPIPAVAEMNPNVNVFVPPRDGFSGPAPRKSKLICEATNFTPKPITVSWLKDGKLVESGFTTDPVTIENKGSTPQTYKVISTLTISEIDWLNLNVYTCRVDHRGLTFLKNVSSTCAASPSTDILTFTIPPSFADIFLSKSANLTCLVSNLATYETLNISWASQSGEPLETKIKIMESHPNGTFSAKGVASVCVEDWNNRKEFVCTVTHRDLPSPQKKFISKPNEVHKHPPAVYLLPPAREQLNLRESATVTCLVKGFSPADISVQWLQRGQLLPQEKYVTSAPMPEPGAPGFYFTHSILTVTEEEWNSGETYTCVVGHEALPHLVTERTVDKSTGKPTLYNVSLIMSDTGGTCY

SEQ ID NO: 18 - protein, 455 aa, Artificial Sequence
  Heavy chain4 mouse IgM variable, mouse IgM CH1, human IgG1 hinge, human IgG1 CH2, human IgG1 CH3
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 454 aa, Artificial Sequence
  Heavy chain5 mouse IgM variable, mouse IgM CH1, mouse IgG1 hinge, human IgG1 CH2, human IgG1 CH3
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSESQSFPNVFPLVSCESPLSDKNLVAMGCLARDFLPSTISFTWNYQNNTEVIQGIRTFPTLRTGGKYLATSQVLLSPKSILEGSDEYLVCKIHYGGKNRDLHVPVPRDCGCKPCICTPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 573 aa, Artificial Sequence
  Heavy chain6 mouse IgM variable, human IgM CH1-4
EVKLVESGGGLVQPGGSLRLSCATSGFTFSDFYMEWVRQPPGKRLEWIAASRNKAKDYTTEYSASVKGRFIVSRDTSQSILYLQMNALRAEDTAIYYCARDGLLEAMDYWGQGTSVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 21 - DNA, 20 bp, Artificial Sequence
  source 1..20: /organism=“Artificial Sequence” /note=“Oct4 qPCR 5′-primer” /mol_type=“unassigned DNA”
ttgggctcgagaaggatgtg

SEQ ID NO: 22 - DNA, 21 bp, Artificial Sequence
  source 1..21: /organism=“Artificial Sequence” /note=“Oct4 qPCR 3′-primer” /mol_type=“unassigned DNA”
tcctctcgttgtgcatagtcg

SEQ ID NO: 23 - DNA, 20 bp, Artificial Sequence
  source 1..20: /organism=“Artificial Sequence” /note=“Sox2 qPCR 5′-primer” /mol_type=“unassigned DNA”
gccctgcagtacaactccat

SEQ ID NO: 24 - DNA, 19 bp, Artificial Sequence
  source 1..19: /organism=“Artificial Sequence” /note=“Sox2 qPCR 3′-primer” /mol_type=“unassigned DNA”
tgccctgctgcgagtagga

SEQ ID NO: 25 - DNA, 19 bp, Artificial Sequence
  source 1..19: /organism=“Artificial Sequence” /note=“Nanog qPCR 5′-primer” /mol_type=“unassigned DNA”
ctcagcctccagcagatgc

SEQ ID NO: 26 - DNA, 23 bp, Artificial Sequence
  source 1..23: /organism=“Artificial Sequence” /note=“Nanog qPCR 3′-primer” /mol_type=“unassigned DNA”
tagatttcattctctggttctgg

SEQ ID NO: 27 - DNA, 19 bp, Artificial Sequence
  source 1..19: /organism=“Artificial Sequence” /note=“Brachuyru qPCR 5′-primer” /mol_type=“unassigned DNA”
gcatgatcaccagccactg

SEQ ID NO: 28 - DNA, 20 bp, Artificial Sequence
  source 1..20: /organism=“Artificial Sequence” /note=“Brachuyru qPCR 3′-primer” /mol_type=“unassigned DNA”
ttaagagctgtgatctcctc

SEQ ID NO: 29 - DNA, 20 bp, Artificial Sequence
  source 1..20: /organism=“Artificial Sequence” /note=“Goosecoid qPCR 5′-primer” /mol_type=“unassigned DNA”
gagaacctcttccaggagac

SEQ ID NO: 30 - DNA, 20 bp, Artificial Sequence
  source 1..20: /organism=“Artificial Sequence” /note=“Goosecoid qPCR 3′-primer” /mol_type=“unassigned DNA”
ttcttaaaccagacctccac

SEQ ID NO: 31 - DNA, 20 bp, Artificial Sequence
  source 1..20: /organism=“Artificial Sequence” /note=“Cyclophilin qPCR 5′-primer” /mol_type=“unassigned DNA”
caatggccaacagagggaag

SEQ ID NO: 32 - DNA, 20 bp, Artificial Sequence
  source 1..20: /organism=“Artificial Sequence” /note=“Cyclophilin qPCR 3′-primer” /mol_type=“unassigned DNA”
ccaaaaacaacatgatgcca

SEQ ID NO: 33 - DNA, 19 bp, Artificial Sequence
  source 1..19: /organism=“Artificial Sequence” /note=“FOXA2 qPCR 5′-primer” /mol_type=“unassigned DNA”
aagacctacaggcgcagct

SEQ ID NO: 34 - DNA, 19 bp, Artificial Sequence
  source 1..19: /organism=“Artificial Sequence” /note=“FOXA2 qPCR 3′-primer” /mol_type=“unassigned DNA”
cgttgaaggagagcgagtg

SEQ ID NO: 35 - DNA, 20 bp, Artificial Sequence
  source 1..20: /organism=“Artificial Sequence” /note=“CER1 qPCR 5′-primer” /mol_type=“unassigned DNA”
cattgggagacctgcaggac

SEQ ID NO: 36 - DNA, 21 bp, Artificial Sequence
  source 1..21: /organism=“Artificial Sequence” /note=“CER1 qPCR 3′-primer” /mol_type=“unassigned DNA”
cccaaagcaaaggttgttctg

SEQ ID NO: 37 - DNA, 50 bp, Artificial Sequence
  source 1..50: /organism=“Artificial Sequence” /note=“Primer 1” /mol_type=“unassigned DNA”
tcatacacatacgatttaggtgacactatagagcggccgcctgcaggaaa

SEQ ID NO: 38 - DNA, 24 bp, Artificial Sequence
  source 1..24: /organism=“Artificial Sequence” /note=“GP921” /mol_type=“unassigned DNA”
gacatttgggaaggactgactctg

SEQ ID NO: 39 - DNA, 20 bp, Artificial Sequence
  source 1..20: /organism=“Artificial Sequence” /note=“GP922” /mol_type=“unassigned DNA”
ctgaaccttcaaggatgctc

SEQ ID NO: 40 - DNA, 20 bp, Artificial Sequence
  source 1..20: /organism=“Artificial Sequence” /note=“GP927” /mol_type=“unassigned DNA”
gctgtaggtgctgtctttgc